(19) United States
(12) Patent Application Publication
(10) Pub. No.: US 2009/0326007 A1
(43) Pub. Date: Dec. 31, 2009

(54) Compounds and methods for modulating sir2 protein activity

(76) Inventors: Ronen Marmorstein, Swarthmore, PA (US); Brandi D. Sanders, Pitman, NJ (US)

Correspondence Address: Jane Massey Licata;Licata & Tyrrell P.C., 66 E. Main Street, Marlton, NJ, 08053, US

(21) Appl. No.: 12/511,679

(22) Filed: Jul. 29, 2009

Related U.S. Application Data
(63) Continuation-in-part of application No. 12/023,287, filed on Jan. 31, 2008.
(60) Provisional application No. 60/887,642, filed on Feb. 1, 2007.

(51) Int. Cl. A61K 31/47

(52) U.S. Cl. 514/311

(57) ABSTRACT

The present invention embraces compounds that modulate the activity of sirtuin deacetylase protein family members and use thereof for modulating Sir2 activity as well as for preventing or treating diseases or disorders associated with Sir2.

### INTRODUCTION

This application is a continuation-in-part application of U.S. patent application Ser. No. 12/023,287, filed Jan. 31, 2008, which claims benefit of priority to U.S. Provisional Patent Application Ser. No. 60/887,642, filed Feb. 1, 2007, the contents of which are incorporated herein by reference in their entireties.

This invention was made in the course of research sponsored by the National Institutes of Health (NIH Grant Nos. CA107107, CA09171, RR-01646, AG031862), National Cancer Institute (Grant No. N01-CO-12400) and the National Science Foundation (NSF Grant No. DMR 0225180). The government has certain rights in the invention.

### BACKGROUND OF THE INVENTION

The class III family of histone deacetylases, silent information regulator 2 (Sir2) proteins, require NAD⁺ to remove an acetyl moiety from the ε-amino group of lysine residues within protein targets (Imai, et al. (2000) Nature 403:795-800; Landry, et al. (2000) Proc. Natl. Acad. Sci. USA 97:5807-5811; Smith, et al. (2000) Proc. Natl. Acad. Sci. USA 97:6658-6663) to yield the deacetylated protein target, nicotinamide, and 2′-O-acetyl-ADP-ribose (Jackson and Denu (2002) J. Biol. Chem. 277:18535-18544; Sauve, et al. (2001) Biochemistry 40:15456-15463). Sir2 proteins are broadly conserved from bacteria to humans (Brachmann, et al. (1995) Genes Dev. 9:2888-2902), and they are able to deacetylate numerous proteins in addition to histones, including acetyl-coA synthetase (Starai, et al. (2002) Science 298:2390-2392), α-tubulin (North, et al. (2003) Mol. Cell 11:437-444), myoD (Fulco, et al. (2003) Mol. Cell 12:51-62), p53, FOXO, Ku70, and NF-κB (Longo and Kennedy (2006) Cell 126:257-268). Their ability to deacetylate such a wide range of substrates has implicated them in playing a stimulatory role in a wide spectrum of biological functions including DNA recombination (Gottlieb and Esposito (1989) Cell 56:771-776) and repair (Bennett, et al. (2001) Mol. Cell Biol. 21:5359-5373), longevity, transcriptional silencing, apoptosis, axonal protection, insulin signaling and fat mobilization (Longo and Kennedy (2006) supra). In addition, increased dosage or expression of Sir2 has been shown to increase lifespan in yeast, worms, flies, and mice, and increased longevity due to a calorie-restricted diet has been shown in most of these animals to be Sir2 dependant (Longo and Kennedy (2006) supra). Conversely, decreased Sir2 activity due to gene deletion or enzyme inhibition shortens yeast lifespan (Kaeberlein, et al. (1999) Genes Dev. 13:2570-2580). Deletion of the mammalian SIRT6 homologue in mice results in genomic instability and an aging-like phenotype (Mostoslavsky, et al. (2006) Cell 124:315-329).

Sir2 proteins couple the removal of the acetyl moiety of acetyl-lysine to cleavage of the high energy glycosidic bond between nicotinamide and ADP-ribose in β-NAD⁺. Nicotinamide, a reaction product and noncompetitive inhibitor of Sir2 proteins (Bitterman, et al. (2002) J. Biol. Chem. 277:45099-45107; Landry, et al. (2000) supra), has also been shown to be a physiological regulator of this family of proteins (Schmidt, et al. (2004) J. Biol. Chem. 279:40122-40129). Yeast cells grown in the presence of nicotinamide show a dramatic reduction in silencing, an increase in rDNA recombination, and a shortening of replicative lifespan (Bitterman, et al. (2002) supra). Nicotinamide can also inhibit Sir2 deacetylation of p53 in mouse embryonic fibroblast cells upon DNA damage (Luo, et al. (2001) Cell 107:137-148), and of histones H3 and H4 in human embryonic kidney cells, which leads to loss of repression by COUP transcription factor interacting proteins 1 and 2 (Senawong, et al. (2003) J. Biol. Chem. 278:43041-43050). Depletion of nicotinamide by overexpression of PCN1, a gene that encodes a nicotinamide deaminase, is sufficient to activate Sir2 and extend yeast lifespan (Anderson, et al. (2003) Nature 423:181-185; Gallo, et al. (2004) Mol. Cell Biol. 24:1301-1312).

Structural studies of the catalytic core region of Sir2 homologues reveal a large and conserved Rossmann fold domain, a smaller and more structurally diverse zinc binding domain, and a series of loops connecting the two domains, forming the catalytic cleft where the substrates bind (Min, et al. (2001) Cell 105:269-279). Several structures of Sir2 proteins in complex with NAD⁺ in a nonproductive conformation (Avalos, et al. (2004) Mol. Cell 13:639-648; Chang, et al. (2002) J. Biol. Chem. 277:34489-34498; Min, et al. (2001) supra; Zhao, et al. (2003) Structure 11:1403-1411) make clear that simultaneous acetyl-lysine binding is required for NAD⁺ to adopt a productive conformation where it is catalytically competent (Avalos, et al. (2004) supra; Zhao, et al. (2004) Proc. Natl. Acad. Sci. USA 101:8563-8568). In this productive conformation, the nicotinamide group of NAD⁺ is bound in a highly conserved “C pocket” (Min, et al. (2001) supra). Furthermore, structures of A. fulgidus Sir2-Af2 bound to NAD⁺ or ADP-ribose and Thermotoga maritima SirTm bound to acetyl-lysine in the presence of high concentrations of nicotinamide show nicotinamide bound in the highly conserved C pocket (Avalos, et al. (2005) Mol. Cell 17:855-868). While an alternate pocket, distinct from the “C pocket”, has been suggested for binding the inhibitory nicotinamide molecule (Zhao, et al. (2004) supra), the nature of this site was not provided.

Compounds which modulate the activity of sirtuin deacetylase protein family members are disclosed in U.S. Patent Application No. 20060025337. While these compounds activate Sir2 enzymes, they do not reverse the Vₘₐₓ decrease associated with nicotinamide.

Needed in the art are compounds that can relieve or enhance the inhibition of Sir2 proteins by nicotinamide. The present invention meets this need in the art by providing the nicotinamide inhibition and base exchange site of Sir2 for use as a target for identifying Sir2 protein effectors.

### SUMMARY OF THE INVENTION

The present invention features pharmaceutical compositions for use in modulating the activity of Sir2 and preventing or treating a disease involving Sir2 protein. In some embodiments, the invention embraces a compound of Table 3 or 4, or an analog thereof.

The invention also features a method for preventing or treating a disease involving Sir2 protein. This method involves administering to a subject in need of treatment an effective amount of a compound of disclosed herein, so that at least one sign or symptom of the disease is prevented, reduced or reversed thereby preventing or treating the disease.

### BRIEF DESCRIPTION OF THE DRAWINGS

FIG. 1 shows the activity, kinetic, and inhibition data for wild-type and mutant yHst2. FIG. 1A shows the relative activity of wild-type and mutant yHst2 enzymes based on their ability to deacetylate a fluorescently-labeled acetylated peptide in duplicate. Error bars represent one SD of experiments done at least in triplicate. FIG. 1B is a Lineweaver-Burk plot describing the NAD⁺ binding kinetics of wild-type yHst2 (diamonds), the yHst2 Il17F and Il17V mutants (squares and triangles, respectively) done in triplicate. FIG. 1C is a Dixon plot describing the nicotinamide inhibition of wild-type yHst2 at varying concentrations of NAD⁺, 15 μM (squares), 25 μM (triangles), 50 μM (diamonds), and 80 μM (circles). Each line is fit to an equation for a noncompetitive inhibitor with data obtained in triplicate. FIG. 1D is a Dixon plot describing the nicotinamide inhibition of yHst2 Il17F with conditions as described in FIG. 1C, but also including 5 μM NAD⁺ (diamonds). FIG. 1E is a Dixon plot describing the nicotinamide inhibition of yHst2 Il17V with conditions as described in FIG. 1D. FIG. 1F is a Lineweaver-Burk plot describing the NAD⁺ binding kinetics of yHst2 Dl18N (diamonds) with data obtained in triplicate. FIG. 1G is a Dixon plot describing the nicotinamide inhibition of yHst2 D18N at varying concentrations of NAD⁺, 50 μM (open diamonds), 100 μM (open squares), 250 μM (solid squares), 450 μM (solid diamonds), 600 μM (open circles), 800 μM (solid circles), 1000 μM (minus signs), 1250 μM (open triangles), 1500 μM (plus signs), 2000 μM (solid triangles).

FIG. 2 shows that transition metal ions are potent inhibitors of Hst2. The amount of deacetylated product formation was measured in the fluorigenic SIRT1 assay for the deacetylase reaction alone (bars with diagonal lines), in the presence of 50 μM indicated compound (open bars) and 500 μM indicated compound (bars with vertical lines). FIG. 2A, Zn²⁺ and Cd²⁺ are inhibitors of Hst2 regardless of whether chloride, sulfate or acetate are the counter ions. FIG. 2B, The transition metals Cu^{1+ or 2+}, Hg²⁺, Ni²⁺, Co²⁺ or Fe^{2+ or 3+} are inhibitors of the Hst2 deacetylase reaction, but the divalent alkali earth metals Ca²⁺ and Mg²⁺ are not.

### DETAILED DESCRIPTION OF THE INVENTION

Compounds have now been identified that relieve nicotinamide inhibition of Sir2 (i.e., Sir2 activators) or inhibit Sir2 activity. In vitro screening assays identified the activators listed in Table 3 and inhibitors listed in Table 4. Of the inhibitors, the most potent inhibitors had IC₅₀ values of 1-10 μM. Structure-activity relationship analysis revealed additional analogs exhibiting effector activity. Accordingly, the present invention embraces compositions containing the Sir2 modulators set forth in Tables 3 and 4, as well as analogs thereof (i.e., as set forth in Tables 6-9) for modulating the activity Sir2 and in the prevention or treatment of disease.

In particular embodiments, the invention embraces a compound of Formula (I):

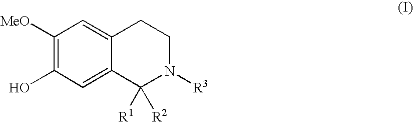

wherein R¹ is a saturated or unsaturated, substituted or unsubstituted C₁₋₅ alkyl group; R² is H or a methyl group; and R³ is a methyl group or a substituted or unsubstituted aryl group.

For the purposes of the present invention, an alkyl group is defined as a straight-chain or branched-chain aliphatic hydrocarbon radical having preferably 1 to 5 carbon atoms (C₁₋₅), especially 1 to 4 carbon atoms (C₁₋₄) or most desirably 1 to 3 carbon atoms (C₁₋₃). Suitable alkyl groups include methyl, ethyl, propyl, isopropyl, butyl, sec-butyl, tert-butyl, and pentyl. An alkyl group of the invention can be saturated or unsaturated, containing at least one double bond or triple bond. In addition, an alkyl group can be unsubstituted or substituted one or more times with, e.g., a halo group (i.e., Cl, Br, F, or I), alkyl, hydroxy, alkoxy, nitro, methylenedioxy, ethylenedioxy, amino, alkylamino, dialkylamino, hydroxyalkyl, hydroxyalkoxy, carboxy, cyano, acyl, alkoxycarbonyl, alkylthio, alkylsulphinyl, alkylsulphonyl, phenoxy, and acyloxy (e.g., acetoxy). In particular embodiments, R¹ is an unsaturated alkyl substituted with an alkoxy group.

Alkoxyl means alkyl-O-groups in which the alkyl portion preferably has 1 to 6 carbon atoms, especially 1 to 4 carbon atoms, or desirably 1 to 2 carbon atoms. Suitable alkoxyl groups include methoxy, ethoxy, propoxy, isopropoxy, isobutoxy, sec-butoxy, pentoxy, hexoxy, heptoxy, and octoxy. Preferred alkoxyl groups are methoxy and ethoxy.

Aryl, as a group or substituent per se refers to an aromatic carbocyclic radical containing 6 to 14 carbon atoms, preferably 6 to 10 carbon atoms, unless indicated otherwise. Suitable aryl groups include phenyl, napthyl and biphenyl. Substituted aryl groups include the above-described aryl groups which are substituted one or more times by halogen, alkyl, hydroxy, alkoxyl, nitro, methylenedioxy, ethylenedioxy, amino, alkylamino, dialkylamino, hydroxyalkyl, hydroxyalkoxy, carboxy, cyano, acyl, alkoxycarbonyl, alkylthio, alkylsulphinyl, alkylsulphonyl, phenoxy, and acyloxy (e.g., acetoxy) groups. Exemplary aryl groups are provided herein.

In particular embodiments, the present invention embraces compounds of Formula (I) as presented in Table 6.

Compounds of Formula (I) are prepared using routine chemical procedures known to those skilled in the art. Likewise, the compounds can be purified to homogeneity using conventional approaches and verified using routine NMR analysis.

Compounds that bind to at least one amino acid residue of the nicotinamide inhibition and base exchange site of Sir2 can be used in a method for modulating (i.e., blocking or inhibiting, or enhancing or activating) a Sir2 protein. Such a method involves contacting a Sir2 protein either in vitro or in vivo with an effective amount of a compound that interacts with at least one amino acid residue of the nicotinamide inhibition and base exchange site of Sir2 so that the activity of the Sir2 protein is modulated. An effective amount of an effector or modulatory compound is an amount which reduces or increases the activity of the Sir2 protein by 10%, 20%, 30%, 40%, 50%, 60%, 70%, 80%, 90% or 100%. Such activity can be monitored by enzymatic assays detecting activity of the Sir2 protein or by monitoring the expression or activity of proteins which are known to be regulated by Sir2 protein (e.g., hTERT, p53, PML, BCL6, TAF₁68, or CTIP2). According to certain embodiments, the Sir2 effector for modulating Sir2 activity is selected from the compounds set forth in Table 3 or 4, or analogs thereof, e.g. as presented in Tables 7-10.

As will be appreciated by one of skill in the art, modulating the activity of a Sir2 protein can be useful in selectively analyzing Sir2 protein signaling events in model systems as well as in preventing or treating diseases and disorders involving Sir2 protein. For example, human SirT1 is involved in muscle differentiation, apoptosis, and neurodegeneration and therefore a compound which activates SirT1 will be useful in the prevention or treatment of neurodegenerative diseases such as Alzheimer's disease, amyotrophic lateral sclerosis (ALS), Parkinson's disease, Huntington's disease, and multiple sclerosis.

Sir2 is also involved in senescence, lifespan, and cell proliferation. In this regard, Sir2-activating compounds can be used in methods for treating or preventing a disease or condition induced or exacerbated by cellular senescence in a subject; methods for decreasing the rate of senescence of a subject, e.g., after onset of senescence; methods for extending the lifespan of a subject; methods for treating or preventing a disease or condition relating to lifespan; methods for treating or preventing a disease or condition relating to the proliferative capacity of cells; and methods for treating or preventing a disease or condition resulting from cell damage or death. Moreover, Sir2 protein effectors can be used in the treatment of diabetes, obesity, and cancer. In the case of human cancer, SIRT1 inhibitors could prevent the deacetylation of p53 and allow apoptosis in response to cellular damage (Luo, et al. (2001) Cell 107:137-148; Vaziri, et al. (2001) Cell 107:149-159); inhibit silencing of tumor suppressor genes whose DNA is hypermethylated (Jones & Baylin (2002) Nat. Rev. Genet. 3:415-428); or increase H4-K16 and H3-K9 acetylation at endogenous promoters to induce gene re-expression in breast and colon cancer cells (Pruitt, et al. (2006) PLoS Genet. 2:e40).

Prevention or treatment typically involves administering to a subject in need of treatment a pharmaceutical composition containing an effective of a compound identified in the screening method of the invention. According to certain embodiments, the Sir2 effector used in the prevention or treatment of disease is selected from the compounds set forth in Tables 3 or 4, or analogs thereof. In most cases this will be a human being, but treatment of agricultural animals, e.g., livestock and poultry, and companion animals, e.g., dogs, cats and horses, is expressly covered herein. The selection of the dosage or effective amount of a compound is that which has the desired outcome of preventing, reducing or reversing at least one sign or symptom of the disease or disorder being treated. Such signs or symptoms are well-known in the art and can be monitored by the skilled clinician upon commencement of treatment.

Pharmaceutical compositions of the invention can be in the form of pharmaceutically acceptable salts and complexes and can be provided in a pharmaceutically acceptable carrier and at an appropriate dose. Such pharmaceutical compositions can be prepared by methods and contain carriers which are well-known in the art. A generally recognized compendium of such methods and ingredients is Remington: The Science and Practice of Pharmacy, Alfonso R. Gennaro, editor, 20th ed. Lippincott Williams & Wilkins: Philadelphia, Pa., 2000. A pharmaceutically-acceptable carrier, composition or vehicle, such as a liquid or solid filler, diluent, excipient, or solvent encapsulating material, is involved in carrying or transporting the subject compound from one organ, or portion of the body, to another organ, or portion of the body. Each carrier must be acceptable in the sense of being compatible with the other ingredients of the formulation and not injurious to the subject being treated.

Examples of materials which can serve as pharmaceutically acceptable carriers include sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations. Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

The compositions of the present invention can be administered parenterally (for example, by intravenous, intraperitoneal, subcutaneous or intramuscular injection), topically (including buccal and sublingual), orally, intranasally, intravaginally, or rectally according to standard medical practices.

The selected dosage level will depend upon a variety of factors including the activity of the particular compound of the present invention employed, the route of administration, the time of administration, the rate of excretion or metabolism of the particular compound being employed, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of a compound at levels lower than that required in order to achieve the desired therapeutic effect and gradually increase the dosage until the desired effect is achieved. This is considered to be within the skill of the artisan and one can review the existing literature on a specific compound or similar compounds to determine optimal dosing.

The compounds disclosed herein can be used as is, or used as lead compounds to identify additional, structurally related compounds, analogs or derivatives which activate or inhibit Sir2. For example, the compounds disclosed herein can be modified based upon the SAR analysis herein to include additional substituents (e.g., O, N, S, OH, CH₃, halo groups, phenyl groups, alkyl groups, etc.), remove substituents (e.g., O, N, S, OH, CH₃, halo groups, phenyl groups, alkyl groups, etc.), or substitute groups (e.g., substitute one halo group for another) in order to provide analogs with improved activity and/or efficacy. As with the initial screens, modified compounds or compound analogs or derivatives can be screened via in silico, in vitro, or in vivo methods to determine activity.

In this respect, the nicotinamide inhibition and base exchange site of Sir2 was determined for the Sir2 homologue, yeast Hst2, the amino acid sequence of which is set forth herein as SEQ ID NO:1. Based upon the crystal structure analysis of yHst2 in complex with an acetyl-lysine 16 histone H4 peptide, intermediate analogue ADP-HPD, and nicotinamide, the nicotinamide inhibition and base exchange site of Sir2 was identified as a hydrophobic pocket formed by amino acid residues F44, E64, F67, N116, I117, and F184. The location of these residues in yHst2 and homologues of yHst2 are listed in Table 1.

TABLE 1

| | | GENBANK | Corresponding Amino Acid Residue in | | | | | |
|---|---|---|---|---|---|---|---|---|
| | | Accession | Reference Sequence | | | | | |
| Source | Sir2 | No. | F44 | E64 | F67 | N116 | I117 | F184 |
| S. c. | yHst2 | NP_015310 | F44 | E64 | F67 | N116 | I117 | F184 |
| D. m. | dSir2 | NP_477351 | F241 | Q262 | F265 | N314 | I315 | F401 |
| H. s. | SirT1 | NP_036370 | F273 | Q294 | F297 | N346 | I347 | F414 |
| | SirT2 | AAK51133 | F59 | E79 | F82 | N131 | I132 | F198 |
| | SirT3 | NP_036371 | F157 | E177 | F180 | N129 | I130 | F294 |
| M. m. | SirT1 | NP_062786 | F265 | Q286 | F289 | N338 | I339 | F406 |
| | SirT3 | AAH25878 | F15 | E35 | F38 | N87 | I88 | F152 |
| C. f. | Sir2 | AAZ81418 | F60 | E80 | F83 | N132 | I133 | F199 |
| R. n. | Sir2 | NP_001008369 | F59 | E79 | F82 | N131 | I132 | F198 |

S. c., Saccharomyces cerevisiae;
D. m., Drosophila melanogaster;
H. s., Homo sapiens;
M. m., Mus musculus;
C. f., Canis familiaris;
R. n., Rattus norvegicus.

A Sir2 enzyme or protein of the present invention is intended to include any member of the silent information regulator 2 family of proteins which transfers an ADP-ribose group from NAD⁺ to an acetyl group (or a protein carrier as is the case with a subset of Sir2 proteins) (Frye (1999) Biochem. Biophys. Res. Commun. 260:273-279). As such, a Sir2 protein can be from any source (see, e.g., Sir2 proteins listed in Table 1). A Sir2 protein of the invention can be identified by the presence of the conserved motif Cys-Xaa-Xaa-Cys-(Xaa)₁₅₋₂₀-Cys-Xaa-Xaa-Cys (SEQ ID NO:2), which binds to Zn⁺ ions (Min, et al. (2001) supra). Molecular phylogenetic analysis has shown that eukaryotic Sir2-like proteins can be grouped into four main branches designated classes I-IV (Frye (2000) Biochem. Biophys. Res. Commun. 273:793-98). For example, the seven human sirtuin genes include all four classes: SIRT1, SIRT2, and SIRT3 are class I, SIRT4 is class II, SIRT5 is class III, and SIRT6 and SIRT7 are class IV. In particular embodiments, the Sir2 protein is a class I eukaryotic Sir2.

Because of the involvement of Sir2 proteins in a growing number of cellular processes, Sir2 proteins are therapeutic drug targets for the development of small molecule effectors. The information obtained from the inhibitor-bound Sir2 complex crystal structures of the present invention reveal detailed information which is useful in the design, isolation, screening and determination of potential compounds which modulate the activity of Sir2 family members. Compounds that bind in the nicotinamide binding site, D pocket, and either sterically block the subsequent acetylation reaction or react with the oxocarbenium ion intermediate may act as effective Sir2-specific inhibitors, while compounds that cannot react with the reaction intermediate and do not perturb the acetylation reaction, would function as Sir2 activators by alleviating nicotinamide inhibition. Since endogenous levels of nicotinamide limit Sir2 activity in yeast cells (Sauve, et al. (2005) Mol. Cell 17:595-601), relief of nicotinamide inhibition is a physiologically viable approach to Sir2 activation.

In this regard, the present invention also features a method for identifying a compound which modulates the activity of a silent information regulator 2 (Sir2) protein. The method of the present invention involves designing or screening for a compound which binds to at least one amino acid residue of the nicotinamide inhibition and base exchange site of Sir2 and testing the designed or screened compound for its ability to modulate the activity of the Sir2 protein. The method of the present invention can be carried out using various in silico, in vitro or in vivo assays based on detecting interactions between the nicotinamide inhibition and base exchange site and a test compound.

Compound designed or screened in accordance with the present invention can interact with at least one of the amino acid residues of the nicotinamide inhibition and base exchange site of Sir2 (see Table 1) via various heterogeneous interactions including, but not limited to van der Waals contacts, hydrogen bonding, ionic interactions, polar contacts, or combinations thereof to contribute to the energy of binding. In general, it is desirable that the compound interacts with 2, 3, 4, 5, or 6 of the amino acid residues of the nicotinamide inhibition and base exchange site of Sir2 to enhance the specificity of the compound for one or more Sir2 proteins. In particular embodiments, the compound interacts with amino acid residue 44, 64, 67, 116, 117 or 184 of SEQ ID NO:1.

In accordance with the present invention, molecular design techniques can be employed to design, identify and synthesize chemical entities and compounds, including inhibitory and stimulatory compounds, capable of binding to the nicotinamide inhibition and base exchange site of Sir2 proteins. The structure of the nicotinamide inhibition and base exchange site of Sir2 can be used in conjunction with computer modeling using a docking program such as GRAM, DOCK, HOOK or AUTODOCK (Dunbrack, et al. (1997) Folding&Design 2:27-42) to identify potential modulators of Sir2 proteins (e.g., yHst2, human SirT1 or human SirT2). This procedure can include computer fitting of compounds to the nicotinamide inhibition and base exchange site of Sir2 to ascertain how well the shape and the chemical structure of the compound will complement the nicotinamide inhibition and base exchange site or to compare the compound with the binding of nicotinamide in the nicotinamide inhibition and base exchange site. Computer programs can also be-employed to estimate the attraction, repulsion and stearic hindrance of the Sir2 protein and effector compound. Generally, the tighter the fit, the lower the stearic hindrances, the greater the attractive forces, and the greater the specificity which are important features for a specific effector compound which is more likely to interact with Sir2 proteins rather than other classes of proteins.

Alternatively, a chemical-probe approach can be employed in the design of Sir2 modulators. For example, Goodford ((1985) J. Med. Chem. 28:849) describes several commercial software packages, such as GRID (Molecular Discovery Ltd., Oxford, UK), which probe the nicotinamide inhibition and base exchange site of Sir2 with different chemical probes, e.g., water, a methyl group, an amine nitrogen, a carboxyl oxygen, and a hydroxyl. Favored sites for interaction between the nicotinamide inhibition and base exchange site and each probe are thus determined, and from the resulting three-dimensional pattern of such sites a putative complementary molecule can be generated.

The compounds of the present invention can also be designed by visually inspecting the three-dimensional structure of Sir2 to determine more effective inhibitors or activators. This type of modeling is generally referred to as “manual” drug design. Manual drug design can employ visual inspection and analysis using a graphics visualization program such as “O” (Jones, et al. (1991) Acta Crystallographica Section A A47:110-119).

Initially effector compounds can be selected for their structural similarity to the X, Y and Z constituents of, e.g., nicotinamide or a lead compound disclosed herein by manual drug design. The structural analog thus designed can then be modified by computer modeling programs to better define the most likely effective candidates. Reduction of the number of potential candidates is useful as it may not be possible to synthesize and screen a countless number of compound variations that may have some similarity to known inhibitory molecules. Such analysis has been shown effective in the development of HIV protease inhibitors (Lam, et al. (1994) Science 263:380-384; Wlodawer, et al. (1993) Ann. Rev. Biochem. 62:543-585; Appelt (1993) Perspectives in Drug Discovery and Design 1:23-48; Erickson (1993) Perspectives in Drug Discovery and Design 1:109-128). Alternatively, random screening of additional small molecule libraries could lead to modulators whose activity may then be analyzed by computer modeling as described above to better determine their effectiveness as inhibitors or activators.

Programs suitable for searching three-dimensional databases include MACCS-3D and ISIS/3D (Molecular Design Ltd, San Leandro, Calif.), ChemDBS-3D (Chemical Design Ltd., Oxford, UK), and Sybyl/3 DB Unity (Tripos Associates, St Louis, Mo.). Programs suitable for compound selection and design include, e.g., DISCO (Abbott Laboratories, Abbott Park, Ill.), Catalyst (Bio-CAD Corp., Mountain View, Calif.), and ChemDBS-3D (Chemical Design Ltd., Oxford, UK).

The compounds designed using the information of the present invention can bind to all or a portion of the nicotinamide inhibition and base exchange site of Sir2 and may be more potent, more specific, less toxic and more effective than known inhibitors for Sir2 proteins. The designed compounds can also be less potent but have a longer half-life in vivo and/or in vitro and therefore be more effective at modulating Sir2 protein activity in vivo and/or in vitro for prolonged periods of time. Such designed modulators are useful to inhibit or activate Sir2 protein activity to, e.g., alter p53 activity, apoptosis, lifespan or sensitivity of cells or organisms to stress.

The present invention also provides the use of molecular design techniques to computationally screen small molecule databases for chemical entities or compounds that can bind to Sir2 in a manner analogous to the nicotinamide as defined by the structure of the present invention. Such computational screening can identify various groups which interact with one or more amino acid residues of the nicotinamide inhibition and base exchange site of Sir2 and can be employed to synthesize the modulators of the present invention.

Compounds which can be screened in accordance with the method of the present invention are generally derived from libraries of agents or compounds. Such libraries can contain either collections of pure agents or collections of agent mixtures. Examples of pure agents include, but are not limited to, proteins, polypeptides, peptides, nucleic acids, oligonucleotides, carbohydrates, lipids, synthetic or semi-synthetic chemicals, and purified natural products. Examples of agent mixtures include, but are not limited to, extracts of prokaryotic or eukaryotic cells and tissues, as well as fermentation broths and cell or tissue culture supernates. Databases of chemical structures are also available from a number of sources including Cambridge Crystallographic Data Centre (Cambridge, UK) and Chemical Abstracts Service (Columbus, Ohio). De novo design programs include Ludi (Biosym Technologies Inc., San Diego, Calif.), Sybyl (Tripos Associates) and Aladdin (Daylight Chemical Information Systems, Irvine, Calif.).

Library screening can be performed as disclosed herein and can be performed in any format that allows rapid preparation and processing of multiple reactions. For in vitro screening assays, stock solutions of the test compounds as well as assay components can be prepared manually and all subsequent pipeting, diluting, mixing, washing, incubating, sample readout and data collecting carried out using commercially available robotic pipeting equipment, automated work stations, and analytical instruments for detecting the signal generated by the assay. Examples of such detectors include, but are not limited to, luminometers, spectrophotometers, and fluorimeters, and devices that measure the decay of radioisotopes.

After designing or screening for a compound which binds to at least one amino acid residue of the nicotinamide inhibition and base exchange site of Sir2, the compound is subsequently tested for its ability to modulate the activity of the Sir2 protein. Such testing can be based upon whether the compound modulates the deacetylase activity of Sir2 (e.g., in a histone deacetylase assay), nicotinamide exchange activity, or based on binding activity. To measure binding constants (e.g., K_{d}), any suitable method known to those in the art can be employed including, e.g., BIACORE analysis, isothermal titration calorimetry, ELISA with a known drug on the plate to show competitive binding, or by a deacetylase activity assay. Alternatively, the compound can be co-crystallized with Sir2 to determine the binding characteristics through X-ray crystallography techniques. See, for example, U.S. Pat. No. 7,149,280 which discloses a method for identifying a ligand of a target macromolecule by obtaining an X-ray crystal diffraction pattern of a compound bound to the macromolecule crystal.

In addition, selectivity of a compound for SIRT1 protein can be determined. For example, the results provided herein demonstrate that certain compounds only inhibit SIRT1, and not SIRT2 or SIRT3, whereas other compounds inhibit all three proteins. Thus, in one embodiment, a compound of the invention is selective for SIRT1 and fails to inhibit or weakly inhibits other homologues. In another embodiment, a compound of the invention is selective for SIRT1 and one other homolog of SIRT1, e.g., SIRT2 or SIRT3. In a further embodiment, a compound of the invention modulates two or more SIRT homologues. Selectivity can be determined as described herein or using any other conventional approach routinely used in the art.

To further evaluate the efficacy of a compound, one of skill will appreciate that a model system of any particular disease or disorder involving Sir2 proteins can be utilized to evaluate the adsorption, distribution, metabolism and excretion of a compound as well as its potential toxicity in acute, sub-chronic and chronic studies. For example, the effector or modulatory compound can be tested in an assay for replicative lifespan in Saccharomyces cerevisiae (Jarolim, et al. (2004) FEMS Yeast Res. 5(2):169-77) or for the ability to modulate the health and survival of mice on a high-calorie diet (Baur, et al. (2006) Nature 444(7117):337-42). See also assays disclosed in U.S. Patent Application No. 20060025337.

The invention is described in greater detail by the following non-limiting examples.

### EXAMPLE 1

### Materials and Methods

Protein Preparation, Crystallization and Structure Determination. The 64-residue C-terminal deletion construct of yHst2 (residues 1-294) and point mutants were purified and expressed according to established methods (Zhao, et al. (2003) Nat. Struct. Biol. 10:864-871). Point mutations in yHst2 (1-294) were generated from the pRSET-A plasmid overexpressing the N-terminal His₆-tagged fusion protein with site-directed mutagenesis based on the QUIKCHANGE protocol from STRATAGENE (Papworth, et al. (1996) Strategies 9:3-4).

Crystals of the yHst2/ADP-HPD/H4 and the yHst2 Il17F/carba-NAD⁺/H4 complexes were grown using the vapor diffusion method at room temperature and were obtained by equilibrating about 0.15 mM of the respective complex against a reservoir solution containing 2.0 M (NH₄)₂S0₄, 100 mM Na citrate, pH 5.6, 200 mM K/Na tartrate or 2.0 M (NH₄)₂S0₄ and 100 mM Na citrate, pH 5.5. To obtain nicotinamide bound complex, yHst2/ADP-HPD/H4 crystals were soaked with reservoir solution supplemented with 50 mM nicotinamide. All crystals were flash frozen in reservoir solution supplemented with 25% (vol/vol) glycerol for data collection.

All crystallographic data was collected on the Al beamline at CHESS, and processed with the HKL2000 suite (HKL Research, Charlottesville, Va.). The structures were solved with the program AMoRe (Navaza (1994) Acta Crystallographica Section A A50:157-163) using the yHst2/ADP-ribose/H4 structure (PDB code lSZD) as a molecular replacement model for the yHst2/ADP-HPD/H4 and yHst2/ADP-HPD/H4+nicotinamide complex structures and the yHst2/carba-NAD⁺/H4 structure (PDB code lSZC) as a molecular replacement model for the yHst2 1117F/carba-NAD⁺/H4 complex structure. Structures were refined with CNS (Brunger, et al. (1998) Acta Crystallogr. D Biol. Crystallogr. 54:905-921) with model building with O (Jones, et al. (1991) supra) with the nicotinamide built into IFo-Fc difference density of the yHst2/ADP-HPD/H4+nicotinamide structure at the end of the refinement. The final models were checked with composite-simulated annealing omit maps.

For high throughput screening assays, a plasmid containing full-length human SIRT1* (FL SIRT1) was transformed into C41 (DE3) cells (Avidis), expressed overnight at 15° C. by addition of 1 mM IPTG to cell cultures and yielded 6× histidine-tagged SIRT1. Cells were harvested and lysed by sonication in buffer containing 50 mM Tris, pH 7.5, 200 mM NaCl, 5 mM imidizole, 10 mM β-mercaptoethanol (BME) and 0.1 mg/mL PMSF. Soluble FL SIRT1 was purified by Ni-NTA (Qiagen, Valencia, Calif.) in a buffer containing 50 mM Tris, pH 7.5, 300 mM NaCl, 10 mM BME, 5% glycerol and 30-600 mM imidizole followed by SUPERDEX-200 gel filtration in buffer containing 50 mM Tris, pH 7.5, 150 mM NaCl, and 10 mM BME. FL SIRT1 eluted between the 670 kDa and 158 kDa globular protein standards. Since SIRT1 is known to aggregate after several days at 4° C., the protein was aliquoted and frozen at −80° C. for use in fluorigenic assays.

A construct of isoform II of SIRT2 with additional residues N-terminal to the SIRT2 gene in a pET30a expression vector was overexpressed as an N-terminal, thrombin-cleavable, His₆-tagged fusion protein in Escherichia coli BL21-Gold (DE3) cells, initially grown at 37° C. to exponential phase and induced with 0.5 mM IPTG at 15° C. overnight. Cells harboring SIRT2 were disrupted by sonication in 20 mM Tris, pH 8.5, 500 mM NaCl and 10 mM BME. Soluble SIRT2 was purified using a combination of Ni-NTA resin, followed by overnight thrombin cleavage, Q SEPHAROSE resin and SUPERDEX-200 analytical gel filtration chromatography, where the protein eluted between the 128- and 44-kDa globular protein standards, in a buffer containing 20 mM Tris, pH 8.5, 150 mM NaCl and 10 mM BME. Human recombinant SIRT3 was purchased from Enzo Life Sciences.

Kinetic Assays. All enzymatic assays were carried out at room temperature in a buffer containing 25 mM Tris-HCl, pH 8.0, 137 mM NaCl, 2.7 mM KCl, and 1 mM MgCl using a deacetylase fluorescent activity assay kit (AK-555, Biomol Research Laboratories, Plymouth Meeting, Pa.). One μM protein, and 500 μM of both NAD⁺ and fluorogenic acetyl-lysine substrate were used and reactions were quenched after 15 minutes by the addition of 10 mM nicotinamide inhibitor and fluorogenic-lysine developer. For NAD⁺Kₘ measurements, 1 μM protein, saturating fluorogenic acetyl-lysine substrate (100 μM), and varying concentrations of NAD⁺ (0.5-5000 μM) were used. Data taken in triplicate were fitted to the equation l/v=(Kₘ/Vₘₐₓ)(1/[S])+1/Vₘₐₓ, using a root mean least squares approach in the form of a double reciprocal Lineweaver-Burk plot where the x-intercept is equal to −l/Kₘ. For nicotinamide Kᵢ measurements, 1 μM protein, saturating fluorogenic acetyl-lysine substrate (100 μM) and varying amounts of NAD⁺ (5-2000 μM) and nicotinamide (0-l mM) were used. Data taken in triplicate were fitted to the equation 1/v=(1+Kₘ/[S])/VₘₐₓKᵢᵢ*[I]+1/Vₘₐₓ(1+Kₘ/[S]), using a root mean least squares approach in the form of a Dixon plot for a strict noncompetitive inhibitor where Kᵢᵢ is equal to −x at the −x intercept. The slopes of the Dixon Plot were replotted versus 1/[NAD⁺], and the slope of the corresponding line was set equal to Kₘ/(Vₘₐₓ*Kᵢ), in order to calculate Kᵢ.

High Throughput Screening (HTS). A high throughput screening protocol based on the SIRT1 Fluorimetric Activity Assay/Drug Discovery Kit was developed (BIOMOL International, Inc., Plymouth Meeting, Pa.). Thirty microliter of a master mix containing 0.4 μL of the 5 mM FLUOR DE LYS-SIRT1 deacetylase substrate (BIOMOL), 3.2 μL of 1 mM NAD⁺ (Sigma, St. Louis, Mo.), 0.052 μL of 130 mM nicotinamide (Sigma), and 26.348 μL of assay buffer (25 mM Tris-Cl, pH 8.0, 137 mM NaCl, 2.7 mM KCl, 1 mM MgCl₂) per well was added to each reaction well. One hundred nanoliters of either a DMSO control or a compound from the small molecule screening library was added to each well by pinning. To begin the reaction, 10 μL of an enzyme master mix containing 2 μM Hst2 (1-294) (MW=34859.9 Da) in assay buffer was added to each reaction well. After four hours, a stop/developer solution containing 8 μL of 100 mM nicotinamide, 8 μl of 5× FLUOR DE LYS Developer II Concentrate (5×) (BIOMOL), and 24 μL of assay buffer per well was added to each reaction well. After 45 minutes, each plate was read on a fluorescence plate reader at an excitation wavelength of 360 nm and an emission wavelength of 460 nm. The nicotinamide and the relatively high concentration of substrates were added to facilitate detection of activators and inhibitors simultaneously. The high throughput screen was carried out at the Broad Institute Chemical Biology Platform's screening facility on ˜50,000 small molecules from their ChemDiv3; PKO4; HSC12; SPBio; and BCB03 libraries, which were selected because they contained commercially available compounds with drug-like structures and known activity; diversity-oriented organic synthesis-derived skeletally and stereochemically diverse small molecule analogs of several synthetic pathways; commercially available compounds with known biological activity that are candidates for influencing stem-cell differentiation including COX, NO, adenylate cyclase, and protein kinase effectors; drug-like molecules from the Prestwick and Spectrum commercial libraries; or diverse compound scaffolds synthesized by Broad Chemists and collaborators, respectively. All compounds were screened in duplicate. The fluorescence signal from each well was normalized to the DMSO controls on each 384-well plate and compared to a DMSO control population based on the following equation:

$$Z = \frac{χ - μ}{σ} ,$$

where χ is the fluorescence signal of the well, μ is the mean of the control (DMSO) population and σ is the standard deviation of the control population. Generally, compounds that gave fluorescence signals higher than 3Z or lower than −3Z were considered hits. Approximately 74 compounds (0.14%) were identified as initial hits and requested as cherry picks. The cherry picked compounds were retested with careful controls to show reproducibility of effect, and rule out autofluorescence, developer inhibition and other assay artifacts.

A similar approach was used to screen a small library of 64 Ruthenium-based kinase inhibitors. The assay was carried out in 96-well plates in a total volume of 50 μL with concentration of all reagents as described above excluding nicotinamide. Hit compounds gave relative fluorescence signals lower than −9Z.

IC₅₀ Determination. The compounds determined to be reproducible and artifact free inhibitors of Hst2 were purchased (12, ChemDiv; 13, TimTec; 14, Spectrum Chemicals & Laboratory Products) or synthesized (15) (Taylor & Schreiber (2006) Org. Lett. 8:143-146). IC₅₀ values were then measured using the same fluorigenic assay described above for Hst2 (160 μM NAD⁺, 100 μM FLUOR DE LYS-SIRT1 deacetylase substrate, 1 μM Hst2, 15-minute reaction time) and FL SIRT1 (240 μM, NAD⁺, 200 μM FLUOR DE LYS-SIRT1 deacetylase substrate, 1 μM FL SIRT1, and 15-minute reaction time). All compounds were solubilized in 25 mM DMSO and diluted for use in the fluorimetric assay of no more than 10% final DMSO concentration. The concentrations of the compounds in the IC₅₀ experiment spanned the range of enzyme activity from no inhibition to complete inhibition. The dose-response curves were then fit to one-site competition or sigmoidal-dose response curves as appropriate in GRAPHPAD PRISM (GraphPad Software, La Jolla, Calif.) and the IC₅₀ was determined. Three independent IC₅₀ measurements were performed for each compound and the average and standard deviation are reported. To directly compare the potency of these compounds to other inhibitors identified in the literature, several known sirtuin inhibitors were obtained (sirtinol, Alexis Biochemicals; splitomycin and suramin, BIOMOL International; tenovin-6, Cayman Chemicals; nicotinamide, Sigma; 9 and 10, Interbioscreen, Ltd; Ro 31820, EMD Chemicals; surfactin, Sigma) and IC₅₀ experiments were performed using the same assay conditions as described above. Several compounds (cambinol, surfactin and splitomycin) have been shown to be competitive with one of the reaction substrates. Additionally, the IC₅₀ values of these compounds also were determined in reaction conditions where the concentration of the competitive substrate was reduced to its approximate Kₘ value (10 and 45 μM of acetyl-lysine and 16 and 24 μM of NAD⁺ for Hst2 and FL SIRT1, respectively)

Reversibility Assay. Each of the inhibitor scaffolds identified in the HTS was tested to determine whether they were reversible, slowly reversible or irreversible inhibitors of Hst2. The reversibility of each compound was identified using the fluorigenic assay described herein. First, either the enzyme alone, the enzyme plus DMSO or the enzyme plus 10× the IC₅₀ concentration of an identified inhibitor was incubated at a concentration of 100 μM Hst2 for 30 minutes. The enzyme, enzyme plus DMSO or enzyme plus inhibitor was then diluted 100-fold into reaction buffer containing the FLUOR DE LYS-SIRT1 deacetylase substrate and NAD⁺ at concentrations equal to their approximate Kₘs (25 μM) to initiate the reaction. The reactions were quenched during the linear region of the reaction timecourse (1-30 minutes) with 10 mM nicotinamide. The progress curves were then plotted and compared to the appropriate enzyme control (Copeland (2005) Evaluation of Enzyme Inhibitors in Drug Discovery, 1^{st} Edn, Hoboken, John Wiley & Sons, Inc.).

Structure Activity Relationship (SAR) Analysis. A number of commercially available small molecule databases were searched and analogs of the scaffolds identified in the HTS were obtained. Compounds 5140108, 6959933, 5237467, 7985301, 7988362, 6802623, 6836332, 6978945, and 5366302 were obtained from ChemBridge. Analogs of scaffold 15 were synthesized as previously described (Taylor & Schreiber (2006) supra). All other compounds used in the SAR analysis were obtained from Specs (R&D Chemicals). Each compound was tested in triplicate at 50 and 500 μM for its effect against Hst2 in the fluorigenic assay (1 μM Hst2, 100 μM FLUOR DE LYS-SIRT1 deacetylase substrate and 160 μM NAD⁺, 15-minute reaction time). The resulting deacetylase activity for the three experiments was averaged and reported as a percentage relative to control wells containing no inhibitor. IC₅₀ values were then measured for compounds that showed significant inhibition of Hst2 as exhibited by no activity at a compound concentration of 500 μM by the protocol described above.

Competition Assay. Each of the identified inhibitor scaffolds were characterized with regard to their ability to compete with the FLUOR DE LYS-SIRT1 deacetylase substrate and NAD⁺ for binding to Hst2. The fluorigenic assay described above was used with a fixed enzyme concentration of 1 μM. When competition with NAD⁺ was being tested, the FLUOR DE LYS-SIRT1 deacetylase substrate was held at a constant concentration of 100 μM and the NAD⁺ was titrated from ⅓-5× K_{mNAD+} (5-80 μM). When competition with the FLUOR DE LYS-SIRT1 deacetylase substrate was being tested, the NAD⁺ concentration was held constant at 160 μM and the FLUOR DE LYS-SIRT1 deacetylase substrate was titrated from ⅓-5× the K_{macetyl-lysine} (3-50 μM). The inhibitors were titrated from ˜½-several times their estimated Kᵢ as indicated. The reaction time was 15 minutes, and each condition was tested in duplicate. The Kₘ, k_{cat} and k_{cat}/Kₘ values were determined by direct fit of the data in SIGMAPLOT (Systat Software, Point Richmond, Calif.) to the Michealis-Menten equation. The Kᵢ and Kᵢₛ (the slope and intercept inhibition constants, respectively) and the competition type were also determined by a direct fit in SIGMAPLOT to both partial and full competitive, noncompetitive, uncompetitive and mixed competition models. Best-fit models were determined by several statistical methods including R², AICs and Sy.x as well as empirical evaluation of α and β values.

Radioactive Deacetylase Assay. The radioactive deacetylase assay used herein was based on the previously reported protocol (Landry, et al. (2000) Biochem. Biophys. Res. Commun. 278:685). An 11-mer was synthesized based on the primary sequence of yeast histone H4 centered around Lys16, where the lysine side chain was acetylated using standard solid phase FMOC chemistry (Guy & Fields (1997) Methods Enzymol. 289:67; Wellings & Atherton (1997) Methods Enzymol. 289:44). This substrate peptide (100 μM), Hst2 (1 μM), and NAD⁺ (155 μM) were added to either ³H-NAD⁺ or ¹⁴C-NAD⁺ (5.4 μM) labeled in the nicotinamide moiety. The indicated inhibitor (100 μM) was added to the assay buffer containing substrate and the reaction was allowed to proceed for 1 hour at room temperature. The reaction was quenched by addition of sodium borate, pH 8.0 and incubation on ice. The nicotinamide product was extracted with ethyl-acetate and then added to scintillation fluid and the radioactivity of the appropriate isotope was measured.

Inhibition Assay Against Human SIRT2 and SIRT3. Percent enzymatic activity in the presence of 50 μM (12a, 13a, 14a) or 100 μM (15a, nicotinamide) inhibitors was measured for human SIRT2 and SIRT3 using the same fluorigenic assay described above. Reaction conditions were as follows for SIRT2 (240 μM NAD⁺, 200 μM FLUOR DE LYS-SIRT1 deacetylase substrate, 1 μM SIRT2, 15 minute reaction time) and SIRT3 (5 mM NAD⁺, 1 mM FLUOR DE LYS-SIRT1 deacetylase substrate, 1 U/reaction SIRT3, and 15 minute reaction time). Two independent measurements were performed for each compound and the average and standard deviation are reported.

Scaffold Purity Determination by LC-MS. To confirm the purity of the commercially available and synthesized inhibitors, LC-MS was performed. The solid compounds were dissolved in either 50% methanol or 70% acetone and 0.1% formic acid. Samples were injected onto an ORBI-TRAP ESI mass spectrometer in either positive or negative mode and scanned from 125 to 1000 Da.

### EXAMPLE 2

### Structure of Nicotinamide Bound to an yHst2/Acetyllysine/ADP-HPD Ternary Complex

The structure of a ternary complex of yHst2 bound to an acetyl-lysine 16 histone H4 derived peptide and carba-NAD⁺, a non-hydrolysable NAD⁺ analogue (Slama and Simmons (1988) Biochemistry 27:183-193; Slama and Simmons (1989) Biochemistry 28:7688-7694; Zhao, et al. (2004) supra) has been reported. The structure revealed that the acetyl group of the acetyl-lysine substrate hydrogen bonds to the 2′ and 3′ hydroxyl groups of the cyclopentane ring, presumably to help position the nicotinamide group in the highly conserved C pocket for hydrolysis, leaving the acetyl group inappropriately positioned for nucleophilic attack of the l′ carbon. A comparison of this structure with a ternary complex in which ADP-ribose replaces carba-NAD⁺, reveals that the ribose ring is rotated by about 90° relative to its corresponding position in carba-NAD⁺ with the 1′-hydroxyl group of the ADP-ribose ring pointing into another highly conserved, hydrophobic “D pocket” that could accommodate an incoming nicotinamide group for a transglycosidation reaction to reform β-NAD⁺ (Zhao, et al. (2004) supra).

To trap free nicotinamide bound to a relevant sirtuin complex, crystals of a ternary complex containing yHst2, an acetyl-lysine 16 histone H4 peptide and the intermediate analogue ADP-HPD were prepared, and soaked crystals with high concentrations of nicotinamide. ADP-HPD was selected as an intermediate analogue for these studies because of its similarity to the proposed positively charged oxocarbenium ion reaction intermediate that is expected to form directly after cleavage of the glycosidic bond between nicotinamide and ADP-ribose (Slama, et al. (1995) J. Med. Chem. 38:389-393).

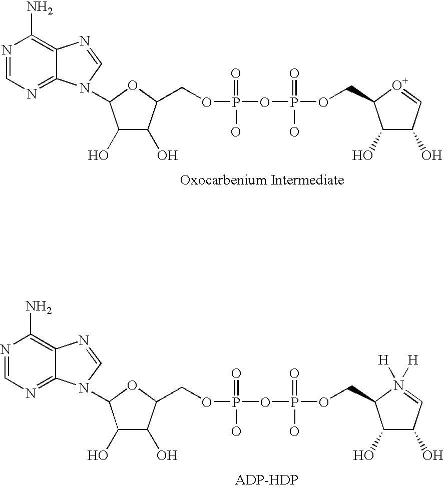

Crystals of the ternary yHst2/ADP-HPD/histone H4 complex in the presence and absence of nicotinamide were isomorphous to the previously described yeast Hst2/ADP-ribose/histone H4 crystals (Zhao, et al. (2004) supra) and formed in the space group P3₂21 containing one molecule per asymmetric unit. The structures were determined by molecular replacement and refined to resolutions of 2.05 Å and 2.00 Å, respectively (Table 2).

TABLE 2

| | | |
|---|---|---|
| | yHst2/ADP- | yHst2 |
| yHst2/ADP- | HPD/H4 + | I117F/carba- |
| HPD/H4 | nicotinamide | NAD⁺/H4 |

Data Collection

| | | | |
|---|---|---|---|
| Space Group | P3₂21 | P3₂21 | P3₂21 |
| Cell parameters, Å | a = b = 106.76, | a = b = 105.94, | a = b = 106.65, |
| | c = 67.7 | c = 67.1 | c = 67.7 |
| Resolution, Å | 50-2.00 | 50-2.05 | 50-2.07 |
| Unique reflections | 28626 | 27580 | 25169 |
| Completeness, %* | 94.2 (94.0) | 99.9 (99.6) | 91.9 (94.5) |
| Multiplicity* | 6.5 (6.1) | 6.1 (5.8) | 6.0 (5.9) |
| I/σ | 53.3 (6.6) | 20.0 (5.6) | 31.5 (10.6) |
| Rmerge, %*^{†} | 6.4 (39.6) | 11.1 (32.0) | 4.6 (11.3) |

Refinement Statistics

| | | | |
|---|---|---|---|
| Rwork, %^{‡} | 22.5 | 21.9 | 22.5 |
| Rfree, %^{§} | 23.3 | 24.6 | 23.3 |
| Number of atoms | | | |
| Protein^{#} | 2312 | 2286 | 2313 |
| H4 peptide | 56 | 61 | 56 |
| Carba-NAD^{+#} | | | 44 |
| ADP-HPD | 35 | 35 | |
| Nicotinamide | | 9 | |
| Water | 116 | 133 | 180 |
| Zn ions^{#} | 1 | 1 | 1 |
| Rms deviations | | | |
| Bond length, Å | 0.007 | 0.006 | 0.007 |
| Bond angles, ° | 1.2 | 1.2 | 1.3 |
| B-factors, Å² | 34.5 | 36.9 | 41.5 |

*Values in parentheses are from the highest resolution shell.
^{#}Values for each molecule in the asymmetric unit.
^{†}R_{merge} = Σ|I − <I>|/Σ<I>.
^{‡}R_{working} = Σ||Fₒ| − |F_{c}||/Σ|Fₒ|.
^{§}R_{free} = Σ_{T}||Fₒ| − |F_{c}||/Σ_{T}^{b}|Fₒ|, where T is a test set of a percentage (5% for all structures) of the total reflections randomly chosen and set aside before refinement.

The structure of the nicotinamide-bound and free complexes were very similar to each other, with an rms deviation of 0.235 Å for C_{α} atoms and the acetyl-lysine and ADP-HPD ligands were also almost identical between these two structures and the previously described ADP-ribose containing complex. The ribose-ring mimic of ADP-HPD was bound in a largely hydrophobic pocket and made only one polar interaction in which a network of water molecules bridged a hydrogen bond between the ring nitrogen of ADP-HPD and N116.

### EXAMPLE 3

### The Nicotinamide Binding Site

The most significant difference between the nicotinamide-bound and free complexes was the presence of a nicotinamide molecule bound in the highly conserved D pocket, adjacent to the β-face of the ADP-HPD molecule in the nicotinamide-bound complex. This pocket was distinct from the C pocket of the nicotinamide moiety of the substrate NAD⁺. It was mostly hydrophobic and formed by residues E64, F184, and F67 around the pyridine ring, and F44 and Il17 proximal to the carboxyamide moiety. Of the residues that formed pocket D, F44, F67, N116 and F184 were strictly conserved, and E64 and 1117 were only conservatively substituted, indicating that this binding site is important for nicotinamide regulation of Sir2 proteins. Since ADP-HPD is analogous to the proposed oxocarbenium intermediate, this conformation of bound nicotinamide is consistent with a nicotinamide molecule binding to the protein complex after the initial nicotinamide cleavage and reacting with the oxocarbenium intermediate, reforming β-NAD⁺. Importantly, the conformation of nicotinamide as described would only be consistent with the exclusive formation of β-NAD⁺.

The nicotinamide molecule had a slightly higher B factor, 74 Å², than the average B factor for the protein atoms in that complex, and the density for the carboxyamide better defined than for the pyridine ring, indicating greater flexibility of the pyridine ring. The carboxyamide oxygen of nicotinamide made a water-mediated hydrogen bond to the backbone nitrogen of I117, while the carboxyamide nitrogen made water-mediated hydrogen bonds to the backbone nitrogen of I117, and the sidechain carbonyl oxygen of N116 and a phosphate oxygen of the ADP-HPD ligand. The pyridine ring of the nicotinamide molecule also made van der Waals interactions with F44 and F67. Residues F44, F67, Nl16, and 1117 are strictly conserved across Sir2 homologues, highlighting the significance of these interactions. Since the nicotinamide pyridine ring makes exclusively van der Waals interactions in the binding pocket and the density for this region is not as clear as for the carboxyamide moiety of nicotinamide, it is contemplated that the ring is free to rotate in the binding pocket. It is believed that when acetyl-lysine is bound, the pyridine ring flips away from the acetyl group and toward the β face of the ribose ring where it can carry out nucleophilic attach of the l′ carbon of the ribose ring of the oxocarbenium ion intermediate. The kinetics of both the cleavage of nicotinamide from NAD⁺ to form the oxocarbenium intermediate, and the attack of the 2′-OH on the acetyl carbonyl carbon are fast as compared to the overall reaction rate (Smith & Denu (2006) Biochemistry 45:272-282). Although enzyme where the general base, R135, is mutated to alanine accumulates the α-1′-O-alkylamidate intermediate, wild-type enzyme accumulates neither the α-1′-O-alkylamidate intermediate or the oxocarbenium intermediate (Smith & Denu (2006) supra), indicating that the presence of nicotinamide in its binding site when or immediately after the intermediate forms will determine whether base exchange or deacetylation chemistry will occur. The structure disclosed herein clearly shows that both nicotinamide and the oxocarbenium intermediate are capable of binding simultaneously to the enzyme active site in a conformation suitable for base exchange chemistry.

In order for nicotinamide to bind the enzyme/oxocarbenium/acetyl-lysine complex, there must be a tunnel that leads from the enzyme active site to solvent in this complex. Indeed, in the nicotinamide bound complex there was a hydrophobic tunnel from the nicotinamide binding site to bulk solvent. The tunnel was formed by residues 37-43 of the flexible β1-α2 loop and was approximately 9×8 Å in diameter, a sufficient size to accommodate a nicotinamide molecule. In the nicotinamide bound complex, the tunnel was occupied by a number of water molecules that made hydrogen bonds mainly to backbone atoms. It is contemplated that after cleavage of NAD⁺ and the formation of the oxocarbenium intermediate, nicotinamide diffuses from solvent or the C pocket through this hydrophobic tunnel to the enzyme active site where it participates in base exchange. This is supported by a superposition of residues that form this tunnel in a number of yHst2 complexes, as substrate or intermediate ternary complexes show an open conformation that would allow nicotinamide to diffuse out or into the tunnel, respectively, while, binary or product complexes show a more closed tunnel.

Based upon the identification of the nicotinamide inhibition and base exchange site of Sir2, in vitro (i.e., in solution) screening assays can also be carried out to identify compounds which selectively bind at the nicotinamide inhibition and base exchange site of Sir2 and inhibit or activate Sir2 protein activity. Selective binding of an agent to the nicotinamide inhibition and base exchange site of Sir2 is achieved by combining a Sir2 protein with NAD⁺, an acetyl-lysine substrate and nicotinamide in solution and determining whether a test compound can either sterically block the subsequent acetylation reaction, react with NAD⁺ or a reaction intermediate, or displace or alleviate nicotinamide inhibition.

### EXAMPLE 4

### Mutations Affecting Nicotinamide Inhibition

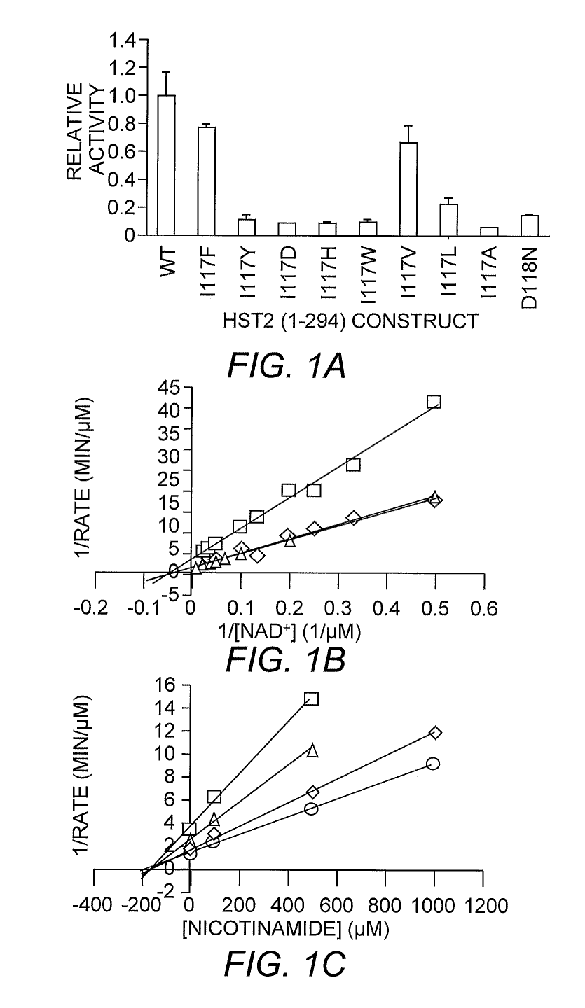

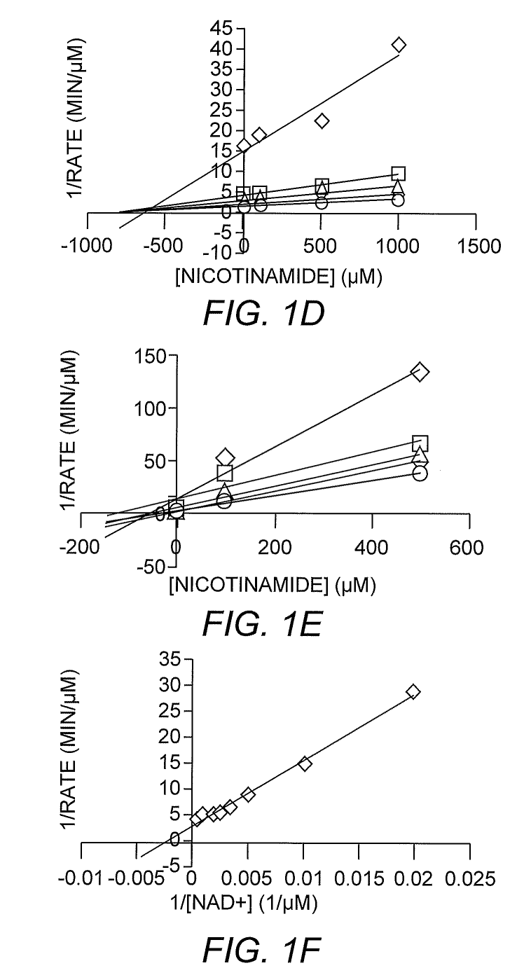

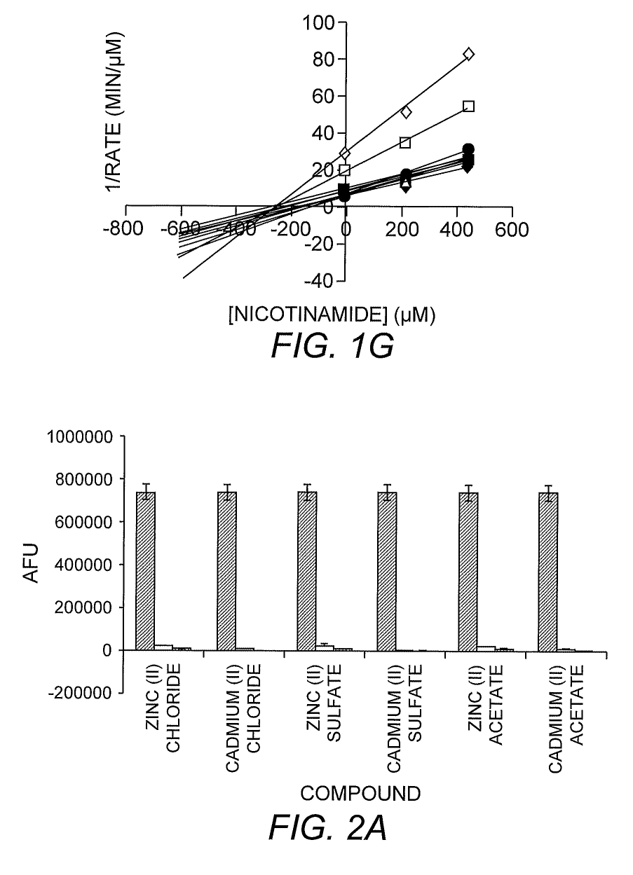

The sidechain of residue I117 sits in the back of the hydrophobic D pocket to which nicotinamide is bound in the yHst2/ADP-HPD/histone H4 nicotinamide bound complex structure, and participates in van der Waals interactions with nicotinamide. To further probe the physiological relevance of this free nicotinamide binding site, yHst2 residue I117 was mutated to several residues and the effect of these mutations on nicotinamide inhibition was measured. Mutations to very large (Y, W), small (A), or charged (H, D) residues rendered the enzyme nearly or completely catalytically inactive, while the mutations I117V or I117F were nearly as active as the wild-type protein (FIG. 1A). Bisubstrate kinetic analysis of the I117V or I117F mutants revealed that they had slightly higher Kₘ values for NAD⁺ relative to the wild-type enzyme, 25.5 μM, 25.8 μM, and 16.1 μM, respectively (FIG. 1B).

Since the I117V and I117F yHst2 mutations had near wild-type activity for deacetylation, their ability to be inhibited by nicotinamide was compared. The Kᵢᵢ for nicotinamide was 1000±30 μM (Kᵢₛ=720±40 μM) for the I117F mutant enzyme, a nearly 6-fold increase over the Kᵢᵢ for nicotinamide of wild-type enzyme, 170±28 μM (Kᵢₛ=140±3 μM) (FIGS. 1C and 1D). This finding is consistent with the bulkier phenylalanine residue partially occluding the nicotinamide binding site within the D pocket. Notably, this increase is much larger than would be expected if free nicotinamide bound in the C pocket because the increased Kₘ for NAD⁺ is less than 2-fold for the I117F mutant enzyme as compared to the wild-type enzyme. Conversely, the Kᵢᵢ for nicotinamide was 65±40 μM (Kᵢₛ=65±1.1 μM) for the I117V mutant enzyme (FIG. 1E), a nearly 2.5-fold decrease over the Kᵢᵢ for nicotinamide of the wild-type enzyme. This result is also consistent with valine being a smaller residue that slightly expands the D pocket, therefore making this pocket slightly more sensitive to nicotinamide inhibition. Together, these functional studies indicate that the D pocket disclosed herein is the site for nicotinamide inhibition.

Nicotinamide has also been shown to inhibit Sir2 enzymes through the same binding pocket that binds the nicotinamide group of NAD⁺, the C pocket (Avalos, et al. (2005) supra). In this study, a D101N mutation in Sir2Tm, a Sir2 homologue from the thermophilic bacterium Thermotoga maritima, resulted in an enzyme that was significantly compromised for both NAD⁺ binding and sensitivity to nicotinamide inhibition. The native aspartic acid residue participates in hydrogen bonding interactions with the amide group of the nicotinamide moiety of NAD⁺, an interaction that is also observed via the corresponding aspartic acid 118 of yHst2 in the yHst2/carba-NAD⁺/acetyl-lysine histone H4 complex. To address the significance of the C pocket in nicotinamide inhibition of yHst2, the corresponding D118N mutation in yHst2 was prepared and its enzymatic properties were characterized. As illustrated in FIG. 1A, the D118N mutant retained approximately 13% of the activity of the wild-type protein, with an apparent Kₘ for NAD⁺ of 453.5 μM, a more than 28-fold increase over the wild-type Kₘ for NAD⁺ (FIGS. 1B and 1F). This increase in Kₘ for NAD⁺ was because the substitution of aspartic acid with asparagine disrupts the important hydrogen bond between the amide group of NAD⁺ and the Asp118 side chain. Subsequently, the Kᵢᵢ for nicotinamide of the D118N mutant was measured. The Kᵢᵢ for nicotinamide for D118N was 180±50 μM (Kᵢ=320±30 μM) (FIG. 1G), very similar to the wild-type value of 170±28 μM. This result indicates that a yHst2, mutation of the C pocket residue, Asn118, has a negligible effect on the sensitivity of the enzyme to nicotinamide inhibition, leading to the conclusion that the D pocket plays a more significant role that the C pocket for nicotinamide inhibition of yHst2 and likely other eukaryotic deacetylases.

### EXAMPLE 5

### Structure of yHst2 I117F in Ternary Complex with Acetyl-Lysine 16 Histone H4 and Carba-NAD⁺

To demonstrate that the sidechain of residue 117 occupies the free nicotinamide binding site, pocket D, and when mutated to phenylalanine, occludes a physiologically relevant nicotinamide binding site, the structure of yHst2 I117F bound to an acetylated histone H4-derived peptide and carba-NAD⁺ was determined. This complex was isomorphous to the previously described wild-type complex (Zhao, et al. (2004) supra) and to the ADP-HPD containing yHst2 complexes described herein. The structure was solved by molecular replacement and refined to 2.0 Å (Table 2).

The yHst2 I117F complex superimposed very well with the wild-type complex. Notably, the hydrogen bonding distance between the carboxyamide carbonyl oxygen of carba-NAD⁺ and the backbone nitrogen of residue 117 was virtually identical, 2.94 Å and 2.89 Å, respectively, between the mutant complex and the wild-type complex, indicating that mutation of residue I117 had a very minor effect on NAD⁺ binding, and would have a similarly minor effect on nicotinamide binding, if free nicotinamide bound in the site previously occupied by the nicotinamide moiety of NAD⁺, as has been proposed (Avalos, et al. (2005) supra; Avalos, et al. (2004) supra).

The structure of the yHst2 Il17F complex showed clear density for the phenylalanine side chain of residue 117 protruding into the free nicotinamide binding pocket described herein, and not into the C pocket that would be occupied by the nicotinamide moiety of NAD⁺. The phenylalanine side chain did not seem to alter the conformation of the nicotinamide moiety of carba-NAD⁺, nor disrupt the interactions this moiety made with any protein residues. Since the yHst2 Il17F/carba-NAD⁺/histone H4 complex was isomorphous with the nicotinamide bound yHst2/ADP-HPD/histone H4 complex, and their protein molecules had an rms deviation of 0.25 Å² for C^{α} atoms, the two complex structures were superimposed. A view of the nicotinamide binding site of the superimposed complexes showed that the I117F sidechain indeed protruded into the free nicotinamide binding site, pocket D, in a way that was incompatible with nicotinamide binding as it was seen in the wild-type enzyme, consistent with the accompanying structural and biochemical studies pointing to the importance of the D pocket for nicotinamide inhibition and base exchange in Sir2 enzymes.

### EXAMPLE 6

### High Throughput Screen for Modulators of Hts2

Based on the identification of the nicotinamide inhibitory and base exchange site of Sir2 deacetylase enzymes, an in vitro screen was carried out to identify yHst2 effectors (potential inhibitors or activators). Approximately 50,000 compounds from several Broad Institute small molecule libraries were screened as potential effectors of the Saccharomyces cerevisiae sirtuin, Hst2. An in vitro fluorescently-based screening assay was developed to simultaneously screen for sirtuin inhibitors and activators. Nicotinamide, a reaction product and inhibitor of the Sir2 deacetylase reaction (Landry, et al. (2000) Proc. Natl. Acad. Sci. USA 97:5807-5811), was added to each reaction well at its approximate IC₅₀ in order to inhibit the deacetylase reaction by half. In this respect, compounds that inhibited or activated deacetylation through Kₘ or Vₘₐₓ effects or increased the net turnover of the reaction through relief of nicotinamide inhibition could be detected.

The same screening approach was used on a kinase inhibitor library composed of Ruthenium-based compounds that were designed as inorganic, staurosporine mimetics (Zhang, et al. (2004) Org. Lett. 6:521-523). Staurosporine is a competitive inhibitor of kinases with respect to ATP (Meggio, et al. (1995) Eur. J. Biochem. 234:317-322). Although sirtuin enzymes do not contain an ATP binding site, they do contain a binding site for the structurally related NAD⁺ molecule. This coupled with the fact that kinase inhibitors have previously been described as both sirtuin activators (Howitz, et al. (2003) Nature 425:191-196) and inhibitors (Trapp, et al. (2006) J. Med. Chem. 49:7307-7316), made this an appropriate library for sirtuin effector screening.

The initial HTS identified 74 potential Hst2 effectors, a hit rate of approximately 0.14%. When retested, several of the compounds did not show reproducible effects, inhibited the trypsin assay developer or were autofluorescent at the wavelengths of the experiment (the putative Hst2 activators). These compounds were not pursued further. Of the remaining compounds, several activators were identified (Table 3) and five classes of inhibitory compounds were identified, namely cadmium acetate or zinc pyrithione (11a and 11b, respectively), 4-(4-Ethyl-phenoxy)-butyric acid 6-oxo-6H-benzo[c]chromen-3-yl ester (12a), 3-(1-Oxo-1,3-dihydro-isoindol-2-yl)-propionic acid (13a), hexachlorophene (14a), 6-Methoxy-1-(3-methoxy-prop-1-ynyl)-2-methyl-1,2,3,4-tetrahydro-isoquinolin-7-ol or 6-Methoxy-2-(4-methoxy-benzyl)-1-(3-methoxy-prop-1-ynyl)-1,2,3,4,6,7-hexahydro-isoquinolin-7-ol (15a or 15b, respectively) (Table 4).

TABLE 3

| | Fold |
|---|---|
| Structure/Chemical Name | Activation |
| 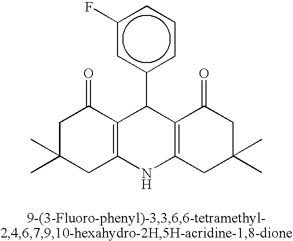 | 2.2 |
| 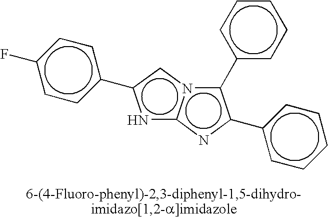 | 2.9 |

TABLE 4

| | | | IC₅₀ |
|---|---|---|---|
| Structure/Chemical Name | Compound | Scaffold | (μM) |
| 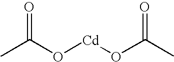 | 11a | 11 | 1.0 ± 0.2 |
| 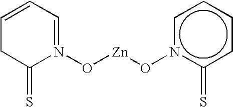 | 11b | 11 | 0.65 ± 0.05 |
| 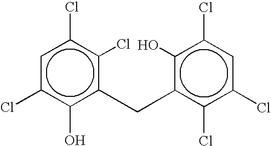 | 14a | 14 | 12.5 ± 0.6 |
| 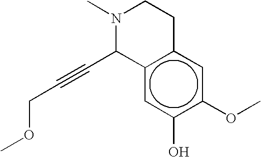 | 15a | 15 | 130 ± 4 |
| 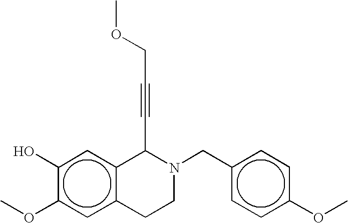 | 15b | 15 | 260 ± 20 |
| 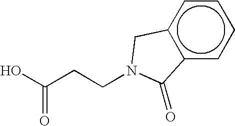 | 13a | 13 | 19.9 ± 0.6 |
| 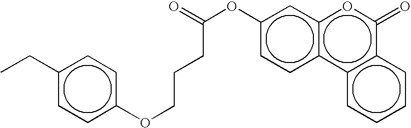 | 12a | 12 | 6.5 ± 1.3 |
| 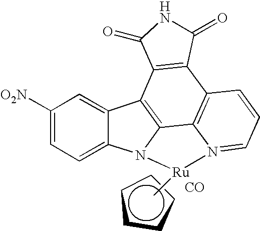 | 16a | 16 | 36 ± 12 |
| 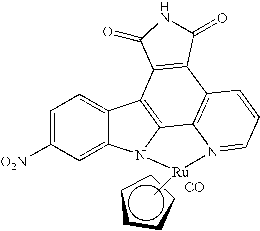 | 16b | 16 | 24 ± 16 |

Each of the inhibitors also showed inhibition of deacetylase activity in an assay using radiolabeled NAD⁺ and an unlabeled acetyl-lysine containing peptide, showing that the presence of a fluorescently labeled acetyl-lysine peptide was not required for inhibition. None of these compounds appeared to be closely related analogs of either reaction substrate. The screen of the 64-member kinase inhibitor library identified two potential Hst2 inhibitors, the structurally related compounds 16a (Pyrido[2,3-a]4-nitropyrrolo[3,4-c]carbazole-5,7(6H)-dione) (cyclopentadienyl) monocarbonyl ruthenium) and 16b ((Pyrido[2,3-a]5-nitropyrrolo[3,4-c]carbazole-5,7(6H)-dione) (cyclopentadienyl)monocarbonyl ruthenium) (Table 4). The similarity of the two organometallic inhibitors among the more diverse library indicated that these compounds mediated a bona fide interaction with Hst2 and were not obtained because of assay artifacts. Both compounds were reproducible inhibitors of Hst2, did not inhibit trypsin, and represented a sixth novel sirtuin inhibitor scaffold.

### EXAMPLE 7

### IC₅₀ Determination against Hst2 and Human SIRT1

Each of the inhibitors inhibited both Hst2 and the full-length human sirtuin, SIRT1, in a dose-dependent manner. The IC₅₀ value defines the concentration of inhibitor required to half-saturate the enzyme population under specific assay conditions and is commonly used as a measure of relative inhibitor potency among compounds. Because IC₅₀ values are typically used to rank-order the potency of validated hits from HTS (Copeland (2005) supra), the IC₅₀ for each of the inhibitors identified in the HTS were determined for both Hst2 and human SIRT1 (Table 5).

TABLE 5

| Compound | Hst2 IC₅₀ (μM) | SIRT1 IC₅₀ (μM) |
|---|---|---|
| Nicotinamide (1) | 91 ± 7 | 250 ± 80 |
| Sirtinol (2) | 48 ± 11 | 120 ± 23 |
| Splitomycin (3) | >600 | >500 |
| Cambinol (4) | >1000 | >600 |
| Tenovin-6 (5) | >600 | >500 |
| Ro 31-8220 (6) | 20.0 ± 0.9 | 25 ± 7 |
| Surfactin (7) | >700 | >600 |
| Suramin (8) | 240 ± 70 | 0.6 ± 0.3 |
| Indole 35 (9) | 1.3 ± 0.1 | 0.18 ± 0.02 |
| Indole 2 (10) | 14.5 ± 0.6 | 0.64 ± 0.06 |
| 11a | 1.0 ± 0.2 | nd |
| 11b | 0.65 ± 0.05 | 2.7 ± 0.2 |
| 12a | 6.5 ± 1.3 | 6.0 ± 0.4 |
| 13a | 19.9 ± 0.6 | 80 ± 5 |
| 14a | 12.5 ± 0.6 | 34 ± 10 |
| 15a | 130 ± 4 | 570 ± 200 |
| 15b | 260 ± 20 | nd |
| 16a | 36 ± 12 | nd |
| 16b | 24 ± 16 | nd |

IC₅₀ data are reported as the mean of and standard deviation of three independent determinations. IC₅₀ values that were not determined are indicated (nd).

The divalent cation containing scaffolds (11a and 11b) were the most potent inhibitors identified in the HTS, with sub-micromolar IC₅₀ values. Several of the newly identified sirtuin inhibitors had low micromolar IC₅₀ values (12a, 13a, 14a, 16a and 16b) while the remaining inhibitors had IC₅₀ values in the mid-micromolar range (15a and 15b). Because changes in solution conditions, such as pH, ionic strength, and temperature and especially the concentration of substrates can alter the measured IC₅₀ value, the IC₅₀ value of several previously identified sirtuin inhibitors (1-10) were determined for comparison, using the same assays conditions as were used for the inhibitors identified in the HTS used in this study. Significant inhibition of Hst2 or SIRT1 was not observed for several of these inhibitors (3-5, 7) under the assay conditions herein, indicating either that these inhibitors are specific for homologues other than the ones tested or the inhibitors are not very potent inhibitors under these assay conditions. Scaffold 11 represented the most potent inhibitor scaffold against Hst2, while the previously identified indoles (9, 10) (Napper, et al. (2005) J. Med. Chem. 48:8045-8054) were the most potent inhibitors for human SIRT1. Interestingly, several of the scaffolds that were identified (12, 13, 14, 16) were more potent than all previously identified inhibitors tested against Hst2, other than the indole compounds, indicating that these scaffolds might be ideal lead molecules for the development of potent and selective sirtuin inhibitors.

Because each of the compounds identified by HTS inhibited both Hst2 and SIRT1, and these sirtuin proteins have very little sequence homology outside of the catalytic core region, these compounds are presumed to bind to the catalytic core region of the proteins and were thus expected to inhibit most sirtuin proteins. To demonstrate this, the extent of inhibition and specificity of the compounds for other sirtuin proteins was determined. This was achieved by testing the ability of the identified compounds to inhibit a panel of human sirtuin homologues. Compound 14a was a potent inhibitor of human SIRT2 and SIRT3, with an apparent IC₅₀ value of less than 50 μM. Compound 12a was a potent inhibitor of SIRT2 but not SIRT3. Compounds 13a and 15a had only weak inhibitory activity against human SIRT2 and SIRT3 (Table 6).

TABLE 6

| SIRT | 1 | 12a | 13a | 14a | 15a |
|---|---|---|---|---|---|
| SIRT2 | 26.0 ± 1.4 | 2.4 ± 0.3 | 89.0 ± 1.8 | 18.5 ± 0.8 | 92.1 ± 1.5 |
| SIRT3 | 42.5 ± 4.8 | 89.0 ± 3.3 | 91.0 ± 4.3 | 39.5 ± 1.6 | 96.4 ± 0.5 |

Data are reported as the percent of residual enzyme activity in the presence of 50 or 100 μM compound relative to the control reaction with no added inhibitor. Data are reported as the average of two independent determinations and the standard deviation of the average.

Human SIRT4 and SIRT7 do not possess in vitro deacetylase activity, and the deacetylase activity of SIRT5 and SIRT6 was too weak under the assay conditions described here to accurately determine the percent of residual enzyme activity. The fact that all of the compounds inhibited SIRT2 and SIRT3 to some extent indicates that these compounds indeed bind in the catalytic core region of sirtuin proteins to exert their inhibitory effects. The varying potencies of these compounds against different human sirtuin homologues indicates that modification of these lead compounds can increase the selectivity of these compounds for a specific homologue relative to the other human sirtuins.

### EXAMPLE 8

### Determining Inhibitor Scaffold Binding Reversibility

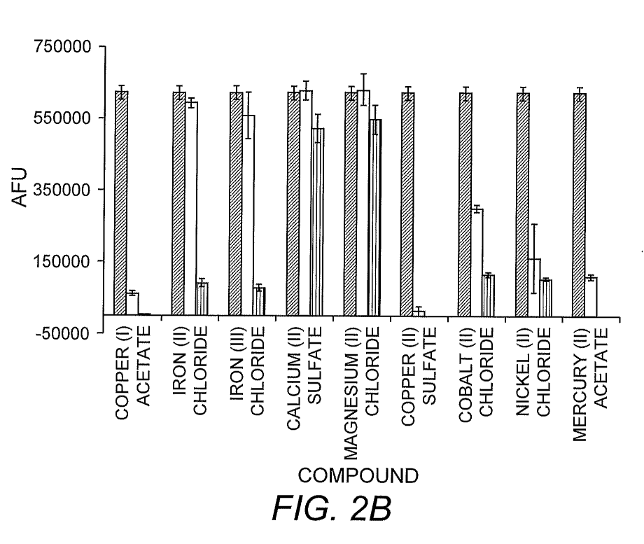

To aid in the determination of the mechanism of inhibition for each newly identified inhibitor and to assay reversibly of inhibitor binding to the enzyme, it was tested whether or not inhibitor binding to the Sir2 enzyme was rapidly reversible, slowly reversible or irreversible. To do this, 100× of the normal assay concentration of enzyme was incubated with 10× the IC₅₀ concentration of each inhibitor. Then, the enzyme was diluted 100-fold into reaction wells with both substrates, and the amount of product formation over time was monitored (FIG. 2). If the enzyme inhibitor was rapidly reversible, the progress curve should be linear with a slope equal to about 91% of the control sample since the final inhibitor concentration will be 0.1×IC₅₀, or about 9% inhibited for a well-behaved concentration-response relationship. If the enzyme was irreversible or very slowly reversible on the time scale of the assay, then only about 9% of residual activity would be monitored after the dilution because the initial incubation of the inhibitor was at a concentration of 10×IC₅₀, or ˜91% inhibition (Copeland (2005) supra). For each of the inhibitor scaffolds identified by HTS, the resulting slopes of the progress curves were all within 9% of the slope of the enzyme only sample, within the error of the experiment and were not near the 9% slope indicative of irreversible inhibitors. The control reversible inhibitor, nicotinamide, actually had the smallest slope with respect to the enzyme control (30%), indicating that this compound was more slowly reversible than any of the compounds identified herein. Although in some specific cases irreversible inhibitors can be useful pharmacologic lead compounds, most lead molecules that function through enzyme inhibition do so through a simple, reversible binding mechanism.

### EXAMPLE 9

### SAR Data for Inhibitors Identified in HTS

A series of structural analogues of the inhibitors identified by HTS were evaluated as inhibitors of Hst2. Compounds 11a and 11b, both contained divalent transition metals. To determine whether the presence of the divalent metal ion or the exact scaffold identified was important for Hst2 inhibition, several compounds containing either divalent zinc or cadmium were tested as possible Hst2 inhibitors (FIG. 2A) using the same fluorigenic assay used in the HTS. Any of the Zn²⁺ or Cd²⁺ compounds inhibited the deacetylase reaction almost completely at 50 μM, indicating that the divalent metal ion and not the counter ion is responsible for Hst2 inhibition. Although metal ion inhibition is common for metalloenzymes (Wells, et al. (1993) Biochemistry 32:1294-1301; Wetterholm, et al. (1994) Arch. Biochem. Abiophys. 311:263-271), Hst2 does not contain an active site metal ion, so the mechanism of metal ion inhibition was not clear, although the metal ion did seem to be acting as a reversible inhibitor and not a general denaturant or precipitant. In order to determine whether the metal ion inhibition was due to a simple charge effect or was dependent upon the valency of the metal ion, the inhibitory effect of several mono-, di-, and trivalent metal salts was tested (FIG. 2B). This analysis indicated that metal ion inhibition was not due to a simple charge effect, as the alkali earth metal cations Ca²⁺ and Mg²⁺ were not Hst2 inhibitors in vitro. However, metal ion inhibition of Hst2 was not contained to Zn²⁺ and Cd²⁺, as each of the divalent transition metal ions tested inhibited Hst2 and the potency of this inhibition seemed to follow a periodic trend (Zn˜Cd>Hg and Zn>Cu>Ni>Co>Fe). Further, the particular valency of the metal ions did not seem to have much of an effect on Hst2 inhibition as Cu¹⁺ and Cu²⁺ and Fe²⁺ and Fe³⁺ inhibited Hst2 to similar extents. Given the unexpected metal inhibition of Hst2, the mechanism of this inhibition could facilitate the elucidation of important aspects of the sirtuin catalytic mechanism.

Following the identification of two compounds with similar scaffolds, 15a and 15b, in the initial HTS as Hst2 inhibitors, several analogs were synthesized, and their effect as inhibitors of Hst2 was evaluated (Table 7).

TABLE 7

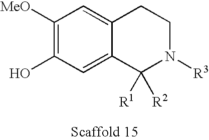

| | Substituent | | | % Activity | |
|---|---|---|---|---|---|
| Compound | R¹ | R² | R³ | 50 μM | 500 μM |
| 7-133 | H | H | 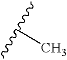 | 100 | 98 |
| 7-52 | 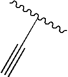 | H | 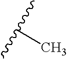 | 100 | 86 |
| 7-56 | 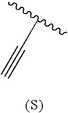 | H | 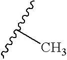 | 96 | 70 |
| 7-57 | 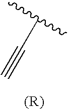 | H | 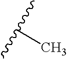 | 97 | 74 |
| 15a | 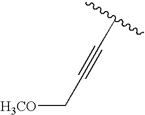 | H | 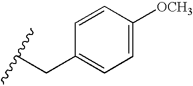 | 76 | 5 |
| 15b | 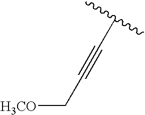 | H | 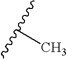 | 92 | 2 |
| 7-128 | 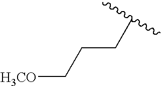 | H | 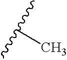 | 99 | 92 |
| 7-129 | 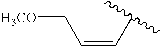 | H | 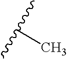 | 97 | 94 |
| 7-123 | 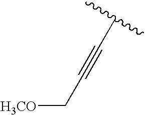 | 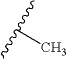 | 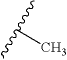 | 100 | 100 |

Data are reported as the percent activity of addition of 50 or 500 μM compound relative to the control reaction with no added inhibitor. Data are reported as the average of three independent determinations, standard deviation of the average≦10%.

Compounds 15a and 15b differ only in their substitution at the isoquinoline nitrogen, and their structural similarity indicates that these small molecules interact with a similar binding site on the enzyme. The IC₅₀ for compound 15a was determined to be about 2-fold lower than that of compound 15b, implying that the enzyme binding site of this scaffold can accommodate, but does not prefer, the larger 4-methoxybenzyl group of 15b. Consequently, all of the structural analogs of 15a and 15b were prepared with a methyl rather than a methoxy-benzyl group on nitrogen. Analysis of the activity of those analogs makes clear that both the alkyne and ether groups in the R¹ position are absolutely required for inhibitory activity. Varying the ligand in the R³ position might be effective in the preparation of more potent related compounds, as these data indicate that substituents in this position can be accommodated by the enzyme active site and may impact the binding interaction with the enzyme.

For the remaining compound identified by HTS, commercially available molecules with similar scaffolds were obtained and evaluated for their effect on Hst2 deacetylase activity. None of the analogs of compound 12a (Table 8) contained a phenyl group in position R⁴ and none of the analogs were as potent as compound 12a, indicating that the phenyl group may be important for inhibitor activity especially since compounds 6802623, 7985301, and 6978945 had significantly reduced potency, although there are other differences between the analogs and the originally identified compound. The length of the carbon chain included in the scaffold (constituents A and B) may be flexible, as compound 5140108 showed some deacetylase inhibition.

TABLE 8

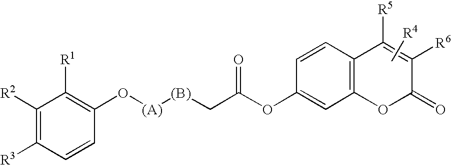

| | |
|---|---|
| | % |
| Substituents | Activity |

| Compound | A | B | R¹ | R² | R³ | R⁴ | R⁵ | R⁶ | 50 | 500 |
|---|---|---|---|---|---|---|---|---|---|---|
| 12a | CH₂ | CH₂ | H | H | CH₂CH₃ | 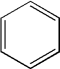 | — | — | <1 | <1 |
| 5140108 | — | — | H | CH₃ | H | — | CH₃ | H | 90 | 13 |
| 6959933 | CH₂ | CH₂ | H | H | OCH₃ | — | CH₃ | H | 93 | 35 |
| 5237467 | — | — | H | H | H | — | CH₃ | H | 91 | 45 |
| 7985301 | CH₂ | CH₂ | CH₃ | H | CH₃ | 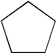 | — | — | 93 | 49 |
| 7988362 | CH₂ | CH₂ | CH₃ | H | CH₃ | — | CH₃ | H | 90 | 51 |
| 6802623 | CH₂ | CH₂ | H | H | CH₂CH₃ | — | CH₃ | H | 86 | 53 |
| 6836332 | CH₂ | CH₂ | H | H | CH₃ | — | CH₃ | H | 99 | 57 |
| 6978945 | CH₂ | CH₂ | H | H | OCH₃ | 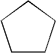 | — | — | 95 | 58 |
| 5366302 | CH₂ | CH₂ | Cl | H | Cl | — | CH₃ | H | 90 | 68 |
| 53378 | CONH | CH₂ | H | H | H | — | CH₃ | CL | 100 | 74 |

Data are reported as the percent activity of addition of 50 or 500 μM compound relative to the control reaction with no added inhibitor. Data are reported as the average of three independent determinations, standard deviation of the average≦10%.

Several analogs of compound 14a were also tested for their ability to inhibit deacetylation by Hst2 (Table 9).

TABLE 9

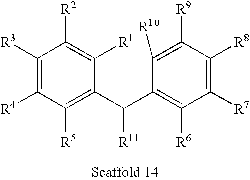

| | | | | | | | | | | | | % | |
|---|---|---|---|---|---|---|---|---|---|---|---|---|---|
| | Substituent | | | | | | | | | | | Activity | |
| Compound | R¹ | R² | R³ | R⁴ | R⁵ | R⁶ | R⁷ | R⁸ | R⁹ | R¹⁰ | R¹¹ | 50 | 500 |
| 14a | Cl | Cl | H | Cl | OH | Cl | Cl | H | Cl | OH | H | <1 | <1 |
| 97252 | H | CH₃ | OH | CH₃ | H | H | CH₃ | OH | CH₃ | H | H | 89 | 43 |
| 97211 | H | H | CF₃ | H | H | H | H | H | H | H | OH | 100 | 81 |
| 72030 | H | H | H | H | H | H | H | OH | H | H | H | 100 | 88 |
| 56413 | H | H | H | NH₂ | H | H | NH₂ | H | H | H | H | 100 | 100 |

| | | | | | | | | | | | | |
|---|---|---|---|---|---|---|---|---|---|---|---|---|
| 43909 | Cl | H | H | H | H | OH | 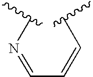 | H | H | NHCOCH₃ | 100 | 100 |

Data are reported as the percent activity of addition of 50 or 500 μM compound relative to the control reaction with no added inhibitor. Data are reported as the average of three independent determinations, standard deviation of the average≦10%.

None of the compounds tested showed significant inhibitory activity against Hst2. The compound with the most inhibitory activity, 97252, had several methyl groups in place of the chloro groups. This indicated that these groups were tolerated in the enzyme active site, but were not ideal for inhibition. The chloro and hydroxyl groups on the originally identified 14a compound probably make important interactions in the enzyme binding site and could be explored individually to determine which are absolutely required for inhibitory effect and which could be changed in order to generate more potent or selective inhibitors.

Several analogs of compound 13a were also tested as potential Hst2 deacetylase inhibitors (Table 10).

TABLE 10

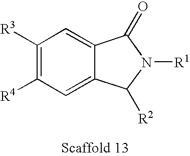

| | | | | | IC₅₀ |
|---|---|---|---|---|---|
| Compound | R¹ | R² | R³ | R⁴ | (μM) |
| 2086, G05 | 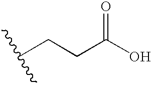 | H | H | H | 19.9 ± 0.5 |
| 39008 | 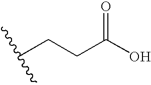 | ═O | Cl | Cl | No Inh. |
| 47054 | 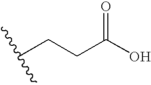 | ═O | 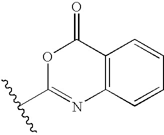 | H | No Inh. |
| 42071 | 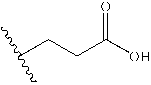 | H | NH₂ | H | No Inh. |
| 427032 | 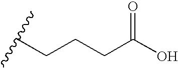 | ═O | H | H | No Inh. |
| 04265 | 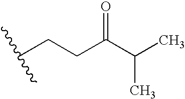 | ═O | H | H | No Inh. |
| 91032 | 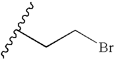 | ═O | H | H | No Inh. |
| 0032 | 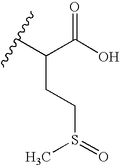 | ═O | H | H | No Inh. |

*indicates which atom of the ligand is directly bound to the scaffold.

None of the compounds tested showed any deacetylase activity against Hst2. Generally, most of the analogs of this compound 13a contained a second ketone functional group and a constituent in the R¹ position of the scaffold of varying carbon chain length. Because the presence of these groups completely abrogated inhibition, the identity of one or both of these groups in the original compound appears to be critical for Hst2 inhibition.

### EXAMPLE 10

### Kinetic Analysis of Inhibition of Hst2 by Selected Inhibitor Scaffolds

To determine the mechanism of inhibition of the compounds identified by HTS and to gain insight as to where these compounds may bind to the enzyme, inhibition modes were determined by varying each of the substrates with the other maintained at a fixed, saturating concentration (Table 11).

TABLE 11

| | | Inhibition | Kᵢ | Kₘ | k_{cat} | k_{cat}/Kₘ |
|---|---|---|---|---|---|---|
| Compound | Substrate | Mechanism | (μM) | (μM) | (min⁻¹) | (min⁻¹/μM⁻¹) |
| 11b | NAD⁺ | Mixed | 0.26 ± 0.08 | 43 ± 5 | 0.87 ± 0.05 | 0.020 |
| | | (full) | | | | |
| | Acetyl- | NC (full) | 0.7 ± 0.1 | 37 ± 10 | 1.3 ± 0.2 | 0.035 |
| | lysine | | | | | |
| 14a | NAD⁺ | NC (full) | 3.9 ± 0.7 | 33 ± 11 | 0.56 ± 0.08 | 0.017 |
| | Acetyl- | Mixed | 2.5 ± 1.0 | 30 ± 9 | 1.33 ± 0.09 | 0.044 |
| | lysine | (full) | | | | |
| 12a | NAD⁺ | NC (full) | 1.2 ± 0.2 | 65 ± 19 | 1.5 ± 0.3 | 0.023 |
| | Acetyl- | NC | 6.3 ± 1.7 | 34 ± 3 | 0.98 ± 0.04 | 0.029 |
| | lysine | (partial) | | | | |
| 13a | NAD⁺ | NC (full) | 30 ± 6 | 70 ± 30 | 0.73 ± 0.13 | 0.010 |
| | Acetyl- | NC (full) | 39 ± 7 | 42 ± 11 | 1.0 ± 0.1 | 0.024 |
| | lysine | | | | | |
| 15a | NAD⁺ | Mixed | 42 ± 17 | 54 ± 17 | 0.82 ± 0.14 | 0.015 |
| | | (full) | | | | |
| | Acetyl- | NC (full) | 43 ± 8 | 32 ± 12 | 1.0 ± 0.2 | 0.031 |
| | lysine | | | | | |

Data reported are the average of two independent determinations, standard deviation of the average as indicated.

With respect to NAD⁺, compounds 12a, 13a, and 14a were best fit to a fully non-competitive inhibition model, with Kᵢ equal to 1.2±0.2, 30±6 and 3.9±0.7 μM, respectively. Compounds 11b and 15a were best fit to a fully mixed inhibition model, with Kᵢ equal to 0.26±0.08 (α=2.2), and 42±17 (α=2.6) μM, respectively. Both of these models imply that the compounds bind to the enzyme regardless of whether or not NAD⁺ is bound, although with reduced affinity in the case of compounds 11b and 15a, indicating that the inhibitors bind to a site distinct from the NAD⁺ binding site. With respect to the acetyl-lysine substrate, compounds 11b, 13a, and 15a were best fit a fully non-competitive inhibition model, with Kᵢ equal to 0.7±0.1, 39±7 and 43±8 μM, respectively. Compound 14a was best fit to a fully mixed inhibition model, with Kᵢ equal to 2.5±1.0 (α=13.4) μM. Finally, compound 12a was best fit to a partial non-competitive inhibition model, with Kᵢ equal to 6.3±1.7 μM. Again, each of these models showed that the newly identified inhibitor compounds bind to both the enzyme alone and the enzyme plus acetyl-lysine complex, indicating that they bind in a site other than the acetyl-lysine binding site.

What is claimed is:

1. A pharmaceutical composition comprising a compound of Formula (I): 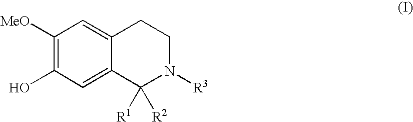
in admixture with a pharmaceutically acceptable carrier, wherein R¹ is a saturated or unsaturated, substituted or unsubstituted C₁₋₅ alkyl; R² is H or a methyl group; and R³ is a methyl group or a substituted or unsubstituted aryl group.

2. A method for preventing or treating a disease involving silent information regulator 2 protein comprising administering to a subject in need of treatment the pharmaceutical composition of claim 1 so that at least one sign or symptom of the disease is prevented, reduced or reversed thereby preventing or treating the disease.

3. The method of claim 2, wherein the disease comprises a neurodegenerative disease, diabetes, obesity, or cancer.

4. A method for preventing or treating a disease involving silent information regulator 2 protein comprising administering to a subject in need of treatment an effective amount of a compound of Table 3 or 4, or an analog thereof, so that at least one sign or symptom of the disease is prevented, reduced or reversed thereby preventing or treating the disease.

5. The method of claim 3, wherein the disease comprises a neurodegenerative disease, diabetes, obesity, or cancer.

6. A method for inhibiting the activity of a silent information regulator 2 (Sir2) protein comprising contacting a Sir2 protein with a compound of Formula (I): 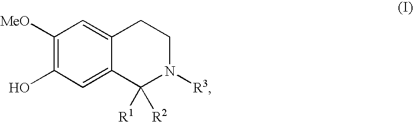
wherein R¹ is a saturated or unsaturated, substituted or unsubstituted C₁₋₅ alkyl; R² is H or a methyl group; and R³ is a methyl group or a substituted or unsubstituted aryl group.

7. A method for modulating the activity of a silent information regulator 2 (Sir2) protein comprising contacting a Sir2 protein with an effective amount of a compound of Table 3 or 4, or an analog thereof, so that the activity of the Sir2 protein is modulated.


SEQUENCE LISTING (1 sequence)

SEQ ID NO: 1 - protein, 357 aa, Saccharomyces cerevisiae
MSVSTASTEMSVRKIAAHMKSNPNAKVIFMVGAGISTSCGIPDFRSPGTGLYHNLARLKLPYPEAVFDVDFFQSDPLPFYTLAKELYPGNFRPSKFHYLLKLFQDKDVLKRVYTQNIDTLERQAGVKDDLIIEAHGSFAHCHCIGCGKVYPPQVFKSKLAEHPIKDFVKCDVCGELVKPAIVFFGEDLPDSFSETWLNDSEWLREKITTSGKHPQQPLVIVVGTSLAVYPFASLPEEIPRKVKRVLCNLETVGDFKANKRPTDLIVHQYSDEFAEQLVEELGWQEDFEKILTAQGGMGDNSKEQLLEIVHDLENLSLDQSEHESADKKDKKLQRLNGHDSDEDGASNSSSSQKAAKE